(19) United States
(12) Patent Application Publication
(10) Pub. No.: US 2009/0012054 A1
(43) Pub. Date: Jan. 8, 2009

(54) Cephem Compounds and Use as Antimicrobial Agents

(75) Inventors: Toshio Yamanaka, Tokyo (JP); Ayako Toda, Tokyo (JP); Hidenori Ohki, Tokyo (JP); Shinya Okuda, Tokyo (JP); Kohji Kawabata, Tokyo (JP); Kenji Murano, Nara (JP); Kazuo Hatano, Tokyo (JP); Shinobu Takeda, Tokyo (JP); Toru Nakai, Tokyo (JP); Masaru Oogaki, Tokyo (JP); Satoshi Inoue, Hiroshima (JP); Keiji Misumi, Hiroshima (JP); Kenji Itoh, Hiroshima (JP)

Correspondence Address: OBLON, SPIVAK, MCCLELLAND MAIER & NEUSTADT, P.C., 1940 DUKE STREET, ALEXANDRIA, VA, 22314, US

(73) Assignees: ASTELLAS PHARMA INC., CHUO-KU (JP); WAKUNAGA PHARMACEUTICAL CO. LTD., OSAKA-SHI (JP)

(21) Appl. No.: 12/280,419

(22) PCT Filed: Mar. 16, 2007
(86) PCT No.: PCT/JP2007/056136

§ 371 Date: Aug. 22, 2008

Related U.S. Application Data
(60) Provisional application No. 60/782,536, filed on Mar. 16, 2006.

(51) Int. Cl. A61K 31/546; C07D 501/16; A61P 31/00

(52) U.S. Cl. 514/202; 544/47

(57) ABSTRACT

The present invention provides a compound of the formula [I]: wherein R1 is lower alkyl, etc. and R2 is hydrogen, etc., or R1 and R2 are bonded together to form lower alkylene; R3 is a group represented by wherein R6 and R7 are independently optionally protected amino, etc.; m and n are independently an integer of 0 to 6; R8 and R9 are independently optionally protected amino, etc., and q and r are independently an integer of 0 to 6, or R8 and R9, together with the adjacent alkylene(s) and the nitrogen atom, form saturated nitrogen-containing heterocycle optionally having substituent(s); R4 is lower alkyl, etc.; and R5 is amino, etc., or a pharmaceutically acceptable salt thereof.

### TECHNICAL FIELD

The present invention relates to new cephem compounds and pharmaceutically acceptable salts thereof. More particularly, the present invention relates to new cephem compounds and pharmaceutically acceptable salts thereof, which have antimicrobial activities, to processes for preparation thereof, to pharmaceutical compositions comprising the same, and to methods for treating infectious diseases in mammals including humans.

### DISCLOSURE OF INVENTION

One object of the present invention is to provide novel cephem compounds and pharmaceutically acceptable salts thereof, which are highly active against a number of pathogenic microorganisms and are suitable for use as medicaments such as antimicrobial agents.

Another object of the present invention is to provide processes for the preparation of said cephem compounds and salts thereof.

A further object of the present invention is to provide pharmaceutical compositions comprising, as an active ingredient, said cephem compounds or their pharmaceutically acceptable salts.

Still further object of the present invention is to provide methods for treating infectious diseases caused by pathogenic microorganisms, which comprises administering said cephem compounds to infected mammals.

Namely, the present invention provides:

[1] A compound of the formula [I] (hereinafter also referred to as compound [I]):

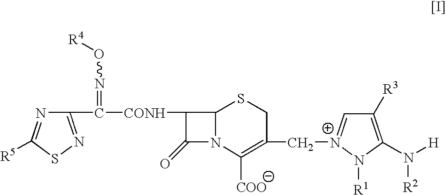

wherein
R¹ is lower alkyl or hydroxy(lower)alkyl, and
R² is hydrogen or amino protecting group, or
R¹ and R² are bonded together to form lower alkylene;
R³ is a group represented by

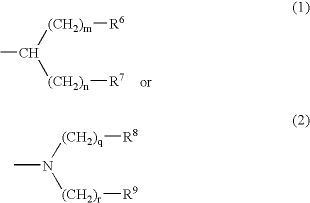

wherein
R⁶ and R⁷ are independently optionally protected amino or optionally protected guanidino, provided that R⁶ and R⁷ are not simultaneously amino groups,
m and n are independently an integer of 0 to 6,
R⁸ and R⁹ are independently optionally protected amino, optionally protected imino(lower)alkylamino, optionally protected guanidino or optionally protected amidino, and
q and r are independently an integer of 0 to 6, or
R⁸ and R⁹, together with the adjacent alkylene(s) and the nitrogen atom, form saturated nitrogen-containing heterocycle optionally having substituent(s),
   R⁴ is lower alkyl optionally substituted with optionally protected carboxy; and
   R⁵ is amino or protected amino,
   or a pharmaceutically acceptable salt thereof.
   [2] The compound of the above-mentioned [1], wherein
   R¹ is lower alkyl or hydroxy(lower)alkyl, and
   R² is hydrogen, aryl(lower)alkyl or acyl, or
   R¹ and R² are bonded together to form lower alkylene;
   R⁴ is lower alkyl optionally substituted with carboxy or esterified carboxy;
   R⁵ is amino, aryl(lower)alkylamino, lower alkanoylamino or lower alkoxycarbonylamino;
   R⁶ and R⁷ are independently guanidino or amino; and
   R⁸ and R⁹ are independently amidino, amino, imino(lower)alkylamino or guanidino, or
   R⁸ and R⁹, together with the adjacent alkylene(s) and the nitrogen atom, form saturated nitrogen-containing heterocycle optionally having substituent(s),
   or a pharmaceutically acceptable salt thereof.
   [3] The compound of the above-mentioned [2], wherein
   R¹ is lower alkyl or hydroxy(lower)alkyl, and
   R² is hydrogen, aryl(lower)alkyl, lower alkanoyl or lower alkoxycarbonyl, or
   R¹ and R² are bonded together to form lower alkylene;
   R⁴ is lower alkyl optionally substituted with carboxy or esterified carboxy;
   R⁵ is amino, aryl(lower)alkylamino or acylamino;
   R⁶ and R⁷ are independently guanidino or amino; and
   R⁸ and R⁹ are independently amidino, amino, imino(lower)alkylamino or guanidino, or
   R⁸ and R⁹; together with the adjacent alkylene(s) and the nitrogen atom, form 5- or 6-membered saturated nitrogen-containing heterocycle optionally having substituent(s),
   or a pharmaceutically acceptable salt thereof.
   [4] The compound of the above-mentioned [3], wherein
   R¹ is lower alkyl,
   R² is hydrogen, or
   R¹ and R² are bonded together to form lower alkylene;
   R⁴ is lower alkyl or carboxy(lower)alkyl;
   R⁵ is amino;
   R⁶ and R⁷ are independently guanidino or amino; and
   R⁵ and R⁹ are independently amidino, amino, imino(lower)alkylamino or guanidino, or
   R⁸ and R⁹, together with the adjacent alkylene(s) and the nitrogen atom, form a group represented by

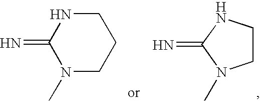

or a pharmaceutically acceptable salt thereof.
[5] A process for preparing a compound of the formula [I]:

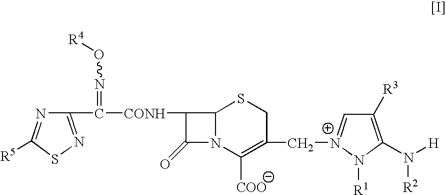

wherein
R¹ is lower alkyl or hydroxy(lower)alkyl, and
R² is hydrogen or amino protecting group, or
R¹ and R² are bonded together to form lower alkylene;
R³ is a group represented by

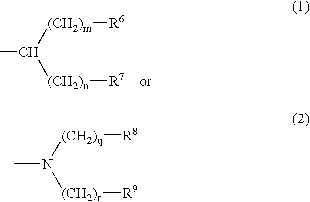

wherein
R⁶ and R⁷ are independently optionally protected amino or optionally protected guanidino, provided that R⁶ and R⁷ are not simultaneously amino groups,
m and n are independently an integer of 0 to 6,
R⁸ and R⁹ are independently optionally protected amino, optionally protected imino(lower)alkylamino, optionally protected guanidino or optionally protected amidino, and
q and r are independently an integer of 0 to 6, or
R⁸ and R⁹, together with the adjacent alkylene(s) and the nitrogen atom, form saturated nitrogen-containing heterocycle optionally having substituent(s),
   R⁴ is lower alkyl optionally substituted with carboxy or protected carboxy; and
   R⁵ is amino or protected amino,
   or a pharmaceutically acceptable salt thereof, which comprises
   (1) reacting a compound of the formula [II]:

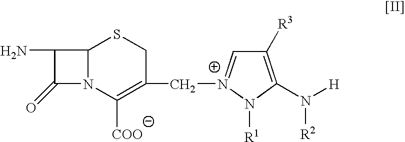

wherein R¹, R² and R³ are each as defined above, or its reactive derivative at the amino group, or a salt thereof with a compound of the formula [III]:

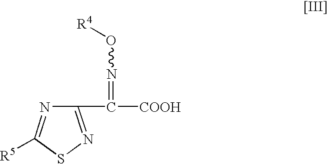

wherein R⁴ and R⁵ are each as defined above, or its reactive derivative at the carboxy group, or a salt thereof to give a compound of the formula [I]:

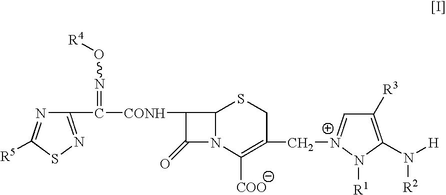

wherein R¹, R², R³, R⁴ and R⁵ are each as defined above, or a salt thereof, or
(2) subjecting a compound of the formula [Ia]:

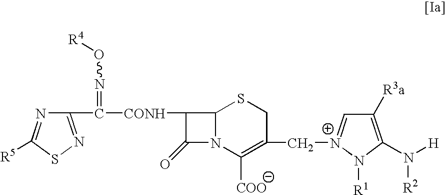

wherein R¹, R², R⁴ and R⁵ are each as defined above, and R³a is a group represented by

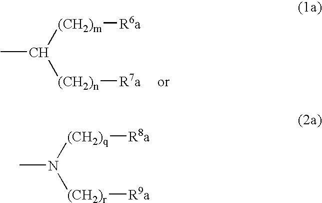

wherein m, n, q and r, are each as defined above,
R⁶a and R⁷a are independently protected amino or protected guanidino, and
R⁸a and R⁹a are independently protected amino, protected imino(lower)alkylamino, protected guanidino or protected amidino, or a salt thereof, to elimination reaction of the amino protecting group to give a compound of the formula [Ib]:

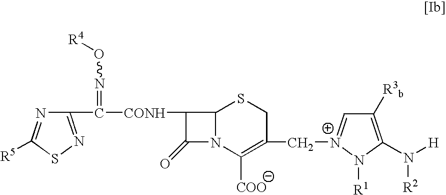

wherein R¹, R², R⁴ and R⁵ are each as defined above, and R³b is a group represented by

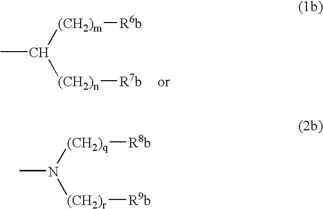

wherein m, n, q and r, are each as defined above,
R⁶b and R⁷b are independently amino or guanidino, and
R⁸b and R⁹b are independently amino, imino(lower)alkylamino, guanidino or amidino, or a salt thereof, or
(3) reacting a compound of the formula [VI]:

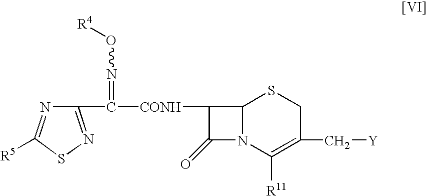

wherein R⁴ and R⁵ are each as defined above, R¹¹ is protected carboxy, and Y is a leaving group, or a salt thereof with a compound of the formula [VII]:

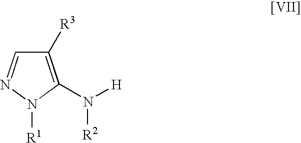

wherein R¹, R² and R³ are each as defined above, or a salt thereof to give a compound of the formula [VIII]:

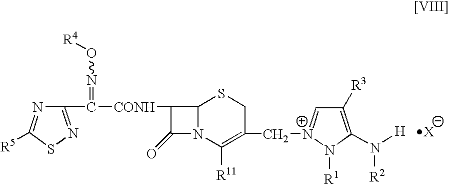

wherein R¹, R², R³, R⁴, R⁵ and R¹¹ are each as defined above, and
X^{\hat{-}} is an anion, or a salt thereof, and
subjecting the compound of the formula [VIII] or a salt thereof to elimination reaction of the carboxy protecting group, to give a compound of the formula [I]:

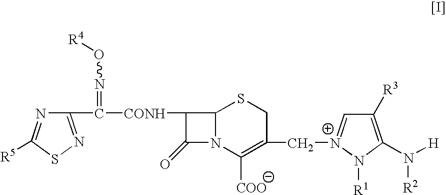

wherein R¹, R², R³, R⁴ and R⁵ are each as defined above, or a salt thereof.
[6] A compound of the above-mentioned [1] or a pharmaceutically acceptable salt thereof for use as a medicament.
[7] A compound of the above-mentioned [1] or a pharmaceutically acceptable salt thereof for use as an antimicrobial agent.
[8] Use of a compound of the above-mentioned [1] or a pharmaceutically acceptable salt thereof for manufacture of a medicament for treating infectious diseases.
[9] A pharmaceutical composition comprising a compound of the above-mentioned [1] or a pharmaceutically acceptable salt thereof in admixture with a pharmaceutically acceptable carrier.
[10] A method for the treatment of infectious diseases which comprises administering a compound of the above-mentioned [1] or a pharmaceutically acceptable salt thereof to a mammal.

As to the object compound [I], the following points are to be noted.

That is, the object compound [I] includes syn isomer (Z form), anti isomer (E form) and a mixture thereof. Syn isomer (Z form) means one geometrical isomer having the partial structure represented by the following formula:

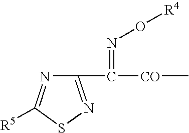

wherein R⁴ and R⁵ are each as defined above, and anti isomer (E form) means the other geometrical isomer having the partial structure represented by the following formula:

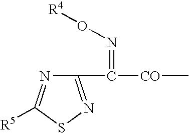

wherein R⁴ and R⁵ are each as defined above, and all of such geometrical isomers and mixture thereof are included within the scope of this invention.

In the present specification and claims, the partial structure of these geometrical isomers and mixture thereof are represented for convenience' sake by the following formula:

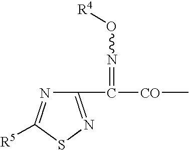

wherein R⁴ and R⁵ are each as defined above.

Another point to be noted is that the pyrazolio moiety of the compound [I] can also exist in the tautomeric form, and such tautomeric equilibrium can be represented by the following formula.

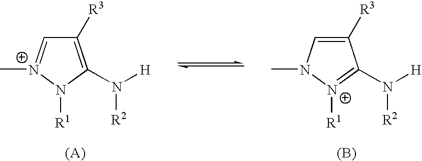

wherein R¹, R² and R³ are each as defined above.

Both of the above tautomeric isomers are included within the scope of the present invention, and in the present specification and claims, however, the object compound [I] is represented for convenience' sake by one expression of the pyrazolio group of the formula (A).

The cephem compound [I] of the present invention can be prepared by the following processes as illustrated in the following.

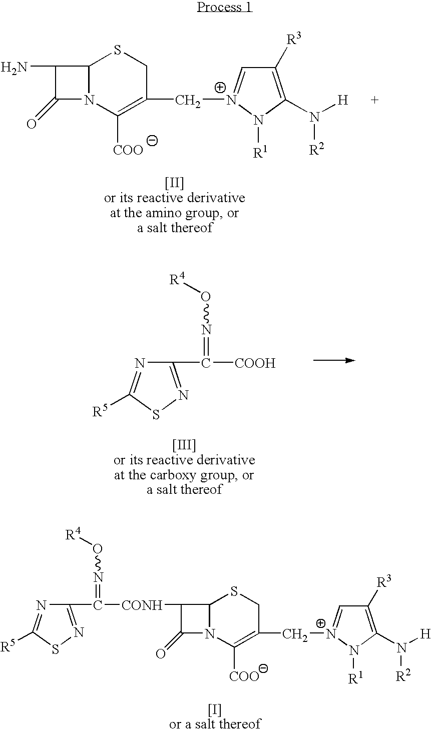

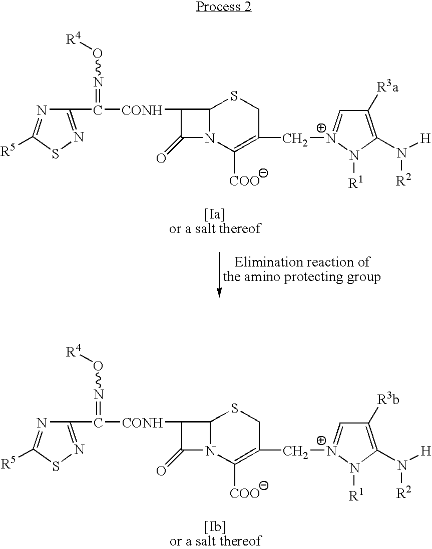

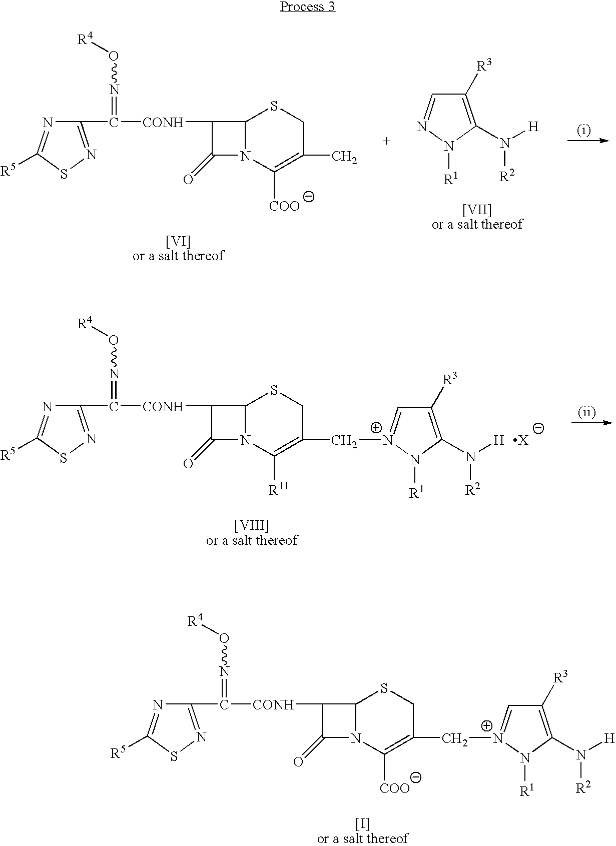

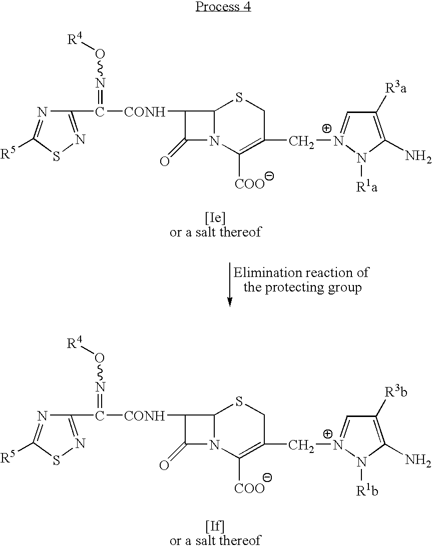

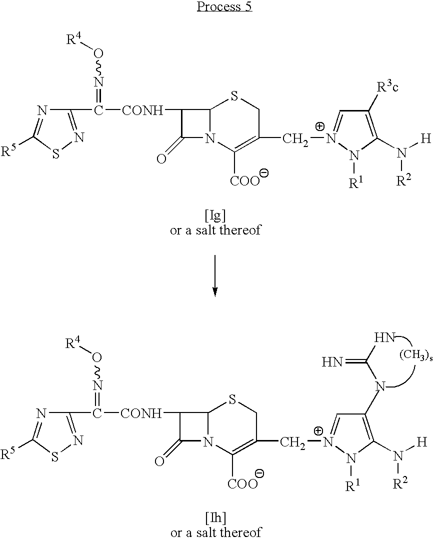

wherein R¹, R², R³, R⁴ and R⁵ are each as defined above,
R¹¹ is protected carboxy,
Y is a leaving group,
X^{\hat{-}} is an anion,

### R³a is

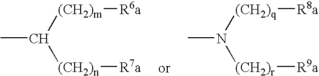 [0026] wherein m, n, q and r are each as defined above, and
R⁶a and R⁷a are independently protected amino or protected guanidino,
m and n are each as defined above, and
R⁸a and R⁹a are independently protected amino, protected imino(lower)alkylamino, protected guanidino or protected amidino,

### R³b is

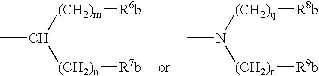 [0031] wherein m, n, q and r are each as defined above, and
R⁶b and R⁷b are independently amino or guanidino,
m and n are each as defined above, and
R⁸b and R⁹b are independently amino, imino(lower)alkylamino, guanidino or amidino,
   R¹a is protected hydroxy(lower)alkyl,
   R¹b is hydroxy(lower)alkyl,

### R³c is

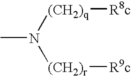 [0036] wherein q and r are each as defined above, and
R⁸c and R⁹c are each amino or amidino, and
   s is 2 or 3.

The starting compounds [II] and [VI] can be prepared by the following processes.

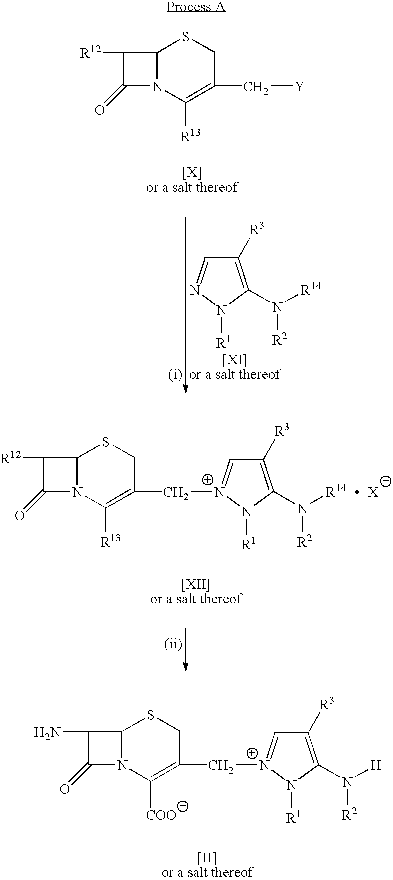

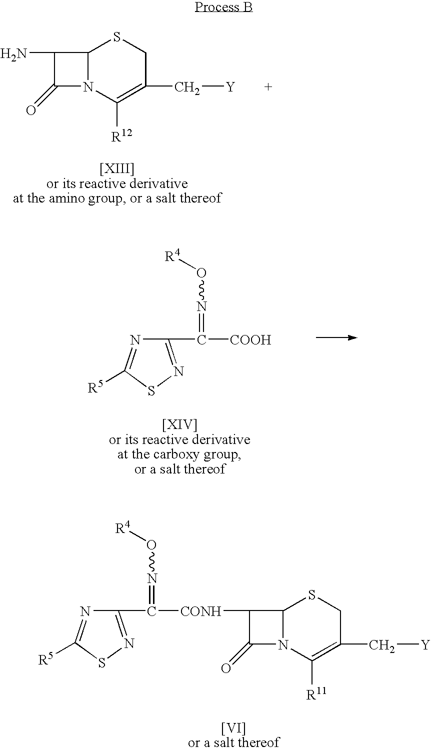

wherein R¹, R², R³, R⁴, R⁵, R¹¹, Y and X^{\hat{-}} are each as defined above,
R¹² is protected amino,
R¹³ is protected carboxy, and
R¹⁴ is amino protecting group.

The starting compounds [VII] and [XI] or salts thereof can be prepared by the methods disclosed in the Preparations described later or similar manners thereto.

In the above and subsequent descriptions of this specification, suitable examples of the various definitions are explained in detail as follows.

The term “lower” is used to mean a group having 1 to 6, preferably 1 to 4 carbon atoms, unless otherwise indicated.

Suitable “lower alkyl” and “lower alkyl” moiety in “hydroxy(lower)alkyl”, “imino(lower)alkylamino”, “aryl(lower)alkyl”, “aryl(lower)alkylamino” and “carboxy(lower)alkyl” include straight or branched C₁₋₆ alkyl, such as methyl, ethyl, propyl, isopropyl, butyl, isobutyl, sec-butyl, tert-butyl, pentyl, tert-pentyl and hexyl, in which more preferred one is C₁₋₄ alkyl.

Suitable “hydroxy(lower)alkyl” includes hydroxy(C₁₋₆)alkyl such as hydroxymethyl, 1-hydroxyethyl, 2-hydroxyethyl, 1-hydroxypropyl, 2-hydroxypropyl, 3-hydroxypropyl, 4-hydroxybutyl, 5-hydroxypentyl and 6-hydroxyhexyl, in which more preferred one is hydroxy(C₁₋₄)alkyl.

Suitable “carboxy(lower)alkyl” includes carboxy(C₁₋₆)alkyl such as carboxymethyl, 1-carboxyethyl, 2-carboxyethyl, 1-carboxypropyl, 2-carboxypropyl, 3-carboxypropyl, 4-carboxybutyl, 5-carboxypentyl and 6-carboxyhexyl, in which more preferred one is carboxy(C₁₋₄)alkyl.

Suitable imino(lower)alkylamino includes imino(C₁₋₆) alkylamino such as iminomethylamino, iminoethylamino, iminopropylamino, iminobutylamino, iminopentylamino and iminohexylamino, in which more preferred one is imino (C₁-C₄) alkylamino.

Suitable “lower alkylene” formed by R¹ and R² includes straight C₁₋₆ alkylene, preferably C₂₋₄ alkylene, such as methylene, ethylene, trimethylene and tetramethylene, in which more preferred one is straight C₂₋₃ alkylene.

Suitable “aryl” moiety in “aryl(lower)alkyl” includes C₆₋₁₂ aryl such as phenyl and naphthyl, in which more preferred one is phenyl.

Suitable “aryl(lower)alkyl” and “aryl(lower)alkyl” moiety includes mono-, di- or triphenyl(lower)alkyl such as benzyl, phenethyl, benzhydryl and trityl.

Suitable “acyl” includes lower alkanoyl [e.g., C₁₋₆ alkanoyl such as formyl, acetyl, propionyl, hexanoyl, pivaloyl, etc.], mono (or di or tri)halo(lower)alkanoyl [e.g., mono (or di or tri)halo(C₁₋₆)alkanoyl such as chloroacetyl, trifluoroacetyl, etc.], lower alkoxycarbonyl [e.g., C₁₋₆ alkoxycarbonyl such as methoxycarbonyl, ethoxycarbonyl, tert-butoxycarbonyl, tert-pentyloxycarbonyl, hexyloxycarbonyl, etc.], carbamoyl, aroyl [e.g., C₇₋₁₃ aroyl such as benzoyl, toluoyl, naphthoyl, etc.], aryl(lower)alkanoyl [e.g., C₆₋₁₂aryl(C₁₋₆)alkanoyl such as phenylacetyl, phenylpropionyl, etc.], aryloxycarbonyl [e.g., C₆₋₁₂ aryloxycarbonyl such as phenoxycarbonyl, naphthyloxycarbonyl, etc.], aryloxy(lower)alkanoyl [e.g., C₆₋₁₂ aryloxy(C₁₋₆)alkanoyl such as phenoxyacetyl, phenoxypropionyl, etc.], arylglyoxyloyl [e.g., C₆₋₁₂ arylglyoxyloyl such as phenylglyoxyloyl, naphthylglyoxyloyl, etc.], aryl(lower)alkoxycarbonyl which optionally having substituent(s) [e.g., C₆₋₁₂ aryloxy-carbonyl optionally having substituent(s) such as benzyloxycarbonyl, phenethyloxycarbonyl, p-nitrobenzyloxycarbonyl, etc.], and the like.

Suitable “lower alkanoyl” and “lower alkanoyl” moiety in “lower alkanoylamino” include straight or branched C₁₋₆ alkanoyl such as formyl, acetyl, propionyl, butyryl, isobutyryl, valeryl, isovaleryl, pivaloyl and hexanoyl, in which more preferred one is C₁₋₄ alkanoyl.

Suitable “lower alkoxy” moiety in “lower alkoxycarbonyl” and “lower alkoxycarbonylamino” includes straight or branched C₁₋₆ alkoxy such as methoxy, ethoxy, propoxy, isopropoxy, butoxy, iso-butoxy, sec-butoxy, tert-butoxy, pentyloxy, tert-pentyloxy and hexyloxy, in which more preferred one is C₁₋₄ alkoxy.

Suitable “saturated nitrogen-containing heterocycle” in “saturated nitrogen-containing heterocycle optionally having substituent(s)” includes 5- or 6-membered saturated nitrogen-containing heterocycles such as pyrimidinyl and imidazolidinyl.

The “substituent” in “saturated nitrogen-containing heterocycle optionally having substituent(s)” may be any substituents, and preferable examples thereof may include imino group and the like.

Suitable “amino protecting group” in “protected amino” includes acyl as mentioned above, substituted or unsubstituted aryl(lower)alkylidene [e.g., benzylidene, hydroxybenzylidene, etc.], aryl(lower)alkyl such as mono-, di- or triphenyl(lower)alkyl [e.g., benzyl, phenethyl, benzhydryl, trityl, etc.], and the like.

Preferable examples of “amino protecting group” include aryl(lower)alkyl and acyl as mentioned above, in which more preferred ones are aryl(lower)alkyl, lower alkanoyl and lower alkoxycarbonyl, and particularly preferred ones are mono-, di- or triphenyl(C₁₋₆)alkyl, C₁₋₆ alkanoyl and (C₁₋₆)alkoxycarbonyl.

Preferable examples of “protected amino” include aryl(lower)alkylamino and acylamino, in which more preferred ones are aryl(lower)alkylamino, lower alkanoylamino and lower alkoxycarbonylamino, and particularly preferred ones are mono-, di- or triphenyl(C₁₋₆)alkylamino, C₁₋₆ alkanoylamino and (C₁₋₆)alkoxycarbonylamino.

As suitable “protecting group” in “protected imino(lower)alkylamino”, “protected guanidino” and “protected amidino”, those exemplified for the aforementioned “amino protecting group” in “protected amino” can be mentioned.

Preferable examples of “protected guanidino” include acylguanidino (monoacylguanidino and diacylguanidino) such as 2,3-bis[(lower)alkoxycarbonyl]guanidino, in which more preferred one is 2,3-bis[(C₁₋₆)alkoxycarbonyl]guanidino [e.g., 2,3-bis(tert-butoxycarbonyl)guanidino].

Preferable examples of “protected amidino” include acylamidino (monoacylamidino and diacylamidino) such as N¹,N²-bis[(lower)alkoxycarbonyl]amidino, in which more preferred one is N¹,N²-bis[(C₁₋₆)alkoxycarbonyl]amidino [e.g., N¹,N²-bis(tert-butoxycarbonyl)amidino].

Suitable “protected hydroxy” in the “protected hydroxy(lower)alkyl” includes acyloxy group, aryl(lower)alkyloxy group, and the like. Suitable “acyl” moiety in the “acyloxy” includes lower alkanoyl [e.g., formyl, acetyl, propionyl, hexanoyl, pivaloyl, etc.], mono(or di or tri)halo(lower)alkanoyl, [e.g., chloroacetyl, trifluoroacetyl, etc.], lower alkoxycarbonyl, [e.g., methoxycarbonyl, ethoxycarbonyl, tert-butoxycarbonyl, tert-pentyloxycarbonyl, hexyloxycarbonyl, etc.], carbamoyl, and the like. Suitable “aryl(lower)alkyl” moiety in the “aryl(lower)alkyloxy” includes aryl(lower)alkyl as mentioned above, and the like.

Suitable “protected carboxy” includes esterified carboxy and the like, and concrete examples of esterified carboxy include lower alkoxycarbonyl [e.g., methoxycarbonyl, ethoxycarbonyl, propoxycarbonyl, isopropoxycarbonyl, butoxycarbonyl, isobutoxycarbonyl, tert-butoxycarbonyl, pentyloxycarbonyl, hexyloxycarbonyl, 1-cyclopropylethoxycarbonyl, etc.] which may have suitable substituent(s), for example, lower alkanoyloxy(lower)alkoxycarbonyl [e.g., acetoxymethoxycarbonyl, propionyloxymethoxycarbonyl, butyryloxymethoxycarbonyl, valeryloxymethoxycarbonyl, pivaloyloxymethoxycarbonyl, 1-acetoxyethoxycarbonyl, 1-propionyloxyethoxycarbonyl, 2-propionyloxyethoxycarbonyl, hexanoyloxymethoxycarbonyl, etc.], lower alkanesulfonyl(lower)alkoxycarbonyl, [e.g., 2-mesylethoxycarbonyl, etc.] or mono(or di or tri)halo(lower)alkoxycarbonyl [e.g., 2-iodoethoxycarbonyl, 2,2,2-trichloroethoxycarbonyl, etc.]; lower alkenyloxycarbonyl [e.g., vinyloxycarbonyl, allyloxycarbonyl, etc.]; lower alkynyloxycarbonyl [e.g., ethynyloxycarbonyl, propynyloxycarbonyl, etc.]; aryl(lower)alkoxycarbonyl which may have suitable substituent(s) [e.g., benzyloxycarbonyl, 4-methoxybenzyloxycarbonyl, 4-nitrobenzyloxycarbonyl, phenethyloxycarbonyl, trityloxycarbonyl, benzhydryloxycarbonyl, bis(methoxyphenyl)methoxycarbonyl, 3,4-dimethoxybenzyloxycarbonyl, 4-hydroxy-3,5-di-tert-butylbenzyloxycarbonyl, etc.]; aryloxycarbonyl which may have suitable substituent(s) [e.g., phenoxycarbonyl, 4-chlorophenoxycarbonyl, tolyloxycarbonyl, 4-tert-butylphenoxycarbonyl, xylyloxycarbonyl, mesityloxycarbonyl, cumenyloxycarbonyl, etc.]; and the like.

Preferable examples of “protected carboxy” include lower alkoxycarbonyl and aryl(lower)alkoxycarbonyl which may have suitable substituent(s), in which more preferred one is (C₁-C₆)alkoxycarbonyl.

Suitable “leaving group” includes halogen [e.g., chlorine, bromine, iodine, etc.] or acyloxy such as arylsulfonyloxy [e.g., benzenesulfonyloxy, tosyloxy, etc.], lower alkylsulfonyloxy [e.g., mesyloxy, etc.], lower alkanoyloxy [e.g., acetyloxy, propionyloxy, etc.], and the like.

Suitable “anion” includes formate, acetate, trifluoroacetate, maleate, tartrate, methanesulfonate, benzenesulfonate, toluenesulfonate, chloride, bromide, iodide, sulfate, phosphate, and the like.

Suitable pharmaceutically acceptable salts of the object compound [I] are conventional non-toxic salts and include, for example, a salt with a base or an acid addition salt such as a salt with an inorganic base, for example, an alkali metal salt [e.g., sodium salt, potassium salt, etc.], an alkaline earth metal salt [e.g., calcium-salt, magnesium salt, etc.], an ammonium salt; a salt with an organic base, for example, an organic amine salt [e.g., trimethylamine salt, triethylamine salt, pyridine salt, picoline salt, ethanolamine salt, triethanolamine salt, dicyclohexylamine salt, N,N′-dibenzylethylenediamine salt, etc.]; an inorganic acid addition salt [e.g., hydrochloride, hydrobromide, sulfate, hydrogensulfate, phosphate, etc.]; an organic carboxylic or sulfonic acid addition salt [e.g., formate, acetate, trifluoroacetate, maleate, tartrate, citrate, fumarate, methanesulfonate, benzenesulfonate, toluenesulfonate, etc.]; and a salt with a basic or acidic amino acid [e.g., arginine, aspartic acid, glutamic acid, etc.].

The preferred embodiments of the compound [I] of the present invention are as follows.

The compound [I], wherein

R¹ is lower alkyl or hydroxy(lower)alkyl, and
R² is hydrogen, aryl(lower)alkyl or acyl, or
R¹ and R² are bonded together and form lower alkylene;
R⁴ is lower alkyl optionally substituted with carboxy or esterified carboxy;
R⁵ is amino, aryl(lower)alkylamino, lower alkanoylamino or lower alkoxycarbonylamino;
R⁶ and R⁷ are independently guanidino or amino; and
R⁸ and R⁹ are independently amidino, amino, imino(lower)alkylamino or guanidino, or
R⁸ and R⁹, together with the adjacent alkylene(s) and the nitrogen atom, form saturated nitrogen-containing heterocycle optionally having substituent(s),
or a pharmaceutically acceptable salt thereof.

The compound [I], wherein

R¹ is lower alkyl or hydroxy(lower)alkyl, and
R² is hydrogen, aryl(lower)alkyl, lower alkanoyl or lower alkoxycarbonyl, or
R¹ and R² are bonded together and form lower alkylene;
R⁴ is lower alkyl optionally substituted with carboxy or esterified carboxy;
R⁵ is amino, aryl(lower)alkylamino or acylamino;
R⁶ and R⁷ are independently guanidino or amino; and
R⁸ and R⁹ are independently amidino, amino, imino(lower)alkylamino or guanidino, or
R⁸ and R⁹, together with the adjacent alkylene(s) and the nitrogen atom, form 5- or 6-membered saturated nitrogen-containing heterocycle optionally having substituent(s),
or a pharmaceutically acceptable salt thereof.

The compound [I], wherein

R¹ is lower alkyl,
R² is hydrogen, or
R¹ and R² are bonded together to form lower alkylene;
R⁴ is lower alkyl or carboxy(lower)alkyl;
R⁵ is amino;
R⁶ and R⁷ are independently guanidino or amino; and
R⁸ and R⁹ are independently amidino, amino, imino(lower)alkylamino or guanidino, or
R⁸ and R⁹, together with the adjacent alkylene(s) and the nitrogen atom, form a group represented by

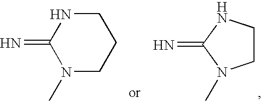

or a pharmaceutically acceptable salt thereof.

The processes for preparing the object compound of the present invention are explained in detail in the following.

### Process 1

The compound [I] or a salt thereof can be prepared by reacting the compound [II] or its reactive derivative at the amino group, or a salt thereof with the compound [III] or its reactive derivative at the carboxy group, or a salt thereof.

Suitable reactive derivative at the amino group of the compound [II] includes Schiff's base type imino or its tautomeric enamine type isomer formed by the reaction of the compound [II] with a carbonyl compound such as aldehyde, ketone and the like; a silyl derivative formed by the reaction of the compound [II] with a silyl compound such as bis(trimethylsilyl)acetamide, mono(trimethylsilyl)acetamide [e.g., N-(trimethylsilyl)acetamide], bis(trimethylsilyl)urea and the like; a derivative formed by the reaction of the compound [II] with phosphorus trichloride or phosgene.

Suitable salts of the compound [II] and its reactive derivative can be referred to the ones as exemplified for the compound [I].

Suitable reactive derivative at the carboxy group of the compound [III] includes an acid halide, an acid anhydride, an activated amide, and an activated ester. A suitable example of the reactive derivatives may be an acid chloride, an acid azide; a mixed acid anhydride with an acid such as substituted phosphoric acid [e.g., dialkylphosphoric acid, phenylphosphoric acid, diphenylphosphoric acid, dibenzylphosphoric acid, halogenated phosphoric acid, etc.], dialkylphosphorous acid, sulfurous acid, thiosulfuric acid, sulfuric acid, alkanesulfonic acid [e.g., methanesulfonic acid, etc.], aliphatic carboxylic acid [e.g., acetic acid, propionic acid, butyric acid, isobutyric acids pivalic acid, pentanoic acid, isopentanoic acid, 2-ethylbutyric acid, trichloroacetic acid, etc.] and aromatic carboxylic acid [e.g., benzoic acid, etc.]; a symmetrical acid anhydride; an activated amide with imidazole, 4-substituted imidazole, dimethylpyrazole, triazole or tetrazole; an activated ester [e.g., cyanomethyl ester, methoxymethyl ester, dimethyliminomethyl [(CH₃)₂N⁺═CH—] ester, vinyl ester, propargyl ester, p-nitrophenyl ester, 2,4-dinitrophenyl ester, trichlorophenyl ester, pentachlorophenyl ester, mesylphenyl ester, phenylazophenyl ester, phenylthio ester, p-nitrophenylthio ester, p-cresylthio ester, carboxymethylthio ester, pyranyl ester, pyridyl ester, piperidyl ester, 8-quinolylthio ester, etc.]; or an ester with an N-hydroxy compound [e.g., N,N-dimethylhydroxylamine, 1-hydroxy-2-(1H)-pyridone, N-hydroxysuccinimide, N-hydroxyphthalimide, N-hydroxy-1H-benzotriazole, etc.]. These reactive derivatives can optionally be selected from them according to the kind of the compound [III] to be used.

Suitable salts of the compound [III] and its reactive derivative can be referred to the ones as exemplified for the compound [I].

The reaction is usually carried out in a conventional solvent such as water, alcohol [e.g., methanol, ethanol, etc.], acetone, dioxane, acetonitrile, chloroform, methylene chloride, ethylene chloride, tetrahydrofuran, ethyl acetate, N,N-dimethylformamide, pyridine or any other organic solvent which does not adversely affect the reaction. These conventional solvents may also be used in a mixture with water.

In this reaction, when the compound [III] is used in free acid form or its salt form, the reaction is preferably carried out in the presence of a conventional condensing agent such as N,N′-dicyclohexylcarbodiimide; N-cyclohexyl-N′-morpholinoethylcarbodiimide; N-cyclohexyl-N′-(4-diethylaminocyclohexyl)carbodiimide; N,N′-diethylcarbodiimide; N,N′-diisopropylcarbodiimide; N-ethyl-N′-(3-dimethylaminopropyl)carbodiimide; N,N′-carbonyl-bis-(2-methylimidazole); pentamethyleneketene-N-cyclohexylimine; diphenylketene-N-cyclohexylimine; ethoxyacetylene; 1-alkoxy-1-chloroethylene; trialkyl phosphite; ethyl polyphosphate; isopropyl polyphosphate; phosphorus oxychloride (phosphoryl chloride); phosphorus trichloride; thionyl chloride; oxalyl chloride; lower alkyl haloformate [e.g., ethyl chloroformate, isopropyl chloroformate, etc.], triphenylphosphine; 2-ethyl-7-hydroxybenzisoxazolium salt; 2-ethyl-5-(m-sulfophenyl)isoxazolium hydroxide intramolecular salt; 1-(p-chlorobenzenesulfonyloxy)-6-chloro-1H-benzotriazole; so-called Vilsmeier reagent prepared by the reaction of N,N-dimethylformamide with thionyl chloride, phosgene, trichloromethyl chloroformate, phosphorus oxychloride, etc.; and the like.

The reaction may also be carried out in the presence of an inorganic or organic base such as an alkali metal bicarbonate, tri(lower)alkylamine, pyridine, N-(lower)alkylmorpholine, N,N-di(lower)alkylbenzylamine, and the like.

The reaction temperature is not critical, and the reaction is usually carried out under cooling to warming.

### Process 2

The compound [Ib] or a salt thereof can be prepared by subjecting the compound [Ia] or a salt thereof to elimination reaction of the amino protecting group.

Elimination reaction is carried out in accordance with a conventional method such as hydrolysis and the like.

The hydrolysis is preferably carried out in the presence of a base or an acid including Lewis acid.

Suitable base includes an inorganic base and an organic base such as an alkali metal [e.g., sodium, potassium, etc.], an alkaline earth metal [e.g., magnesium, calcium, etc.], the hydroxide or carbonate or hydrogencarbonate thereof, trialkylamine [e.g., trimethylamine, triethylamine, etc.], picoline, 1,5-diazabicyclo[4.3.0]non-5-ene, 1,4-diazabicyclo[2.2.2]octane, 1,8-diazabicyclo[5.4.0]undec-7-ene, and the like.

Suitable acid includes an organic acid [e.g., formic acid, acetic acid, propionic acid, trichloroacetic acid, trifluoroacetic acid, etc.], and an inorganic acid [e.g., hydrochloric acid, hydrobromic acid, sulfuric acid, hydrogen chloride, hydrogen bromide, etc.].

The elimination using Lewis acid such as trihaloacetic acid [e.g., trichloroacetic acid, trifluoroacetic acid, etc.], and the like is preferably carried out in the presence of cation trapping agents [e.g., anisole, phenol, etc.].

The reaction is usually carried out in a solvent such as water, alcohol [e.g., methanol, ethanol, etc.], methylene chloride, tetrahydrofuran, a mixture thereof or any other solvent which does not adversely influence the reaction. A liquid base or acid can be also used as a solvent.

The reaction temperature is not critical and the reaction is usually carried out under cooling to warming.

### Process 3-(i)

The compound [VIII] or a salt thereof can be prepared by reacting the compound [VI] or a salt thereof with the compound [VII] or a salt thereof.

Suitable salt of the compounds [VI], [VII] and [VIII] can be referred to the ones as exemplified for the compound [I].

The present reaction may be carried out in a solvent such as water, phosphate buffer, acetone, chloroform, acetonitrile nitrobenzene, methylene chloride, ethylene chloride, formamide, N,N-dimethylformamide, methanol, ethanol, diethyl ether, tetrahydrofuran, dimethyl sulfoxide, or any other organic solvent which does not adversely affect the reaction, preferably in ones having strong polarities. Among the solvents, hydrophilic solvents may be used in a mixture with water. When the compound [VII] is liquid, it can also be used as a solvent.

The reaction is preferably conducted in the presence of a base, for example, an inorganic base such as alkali metal hydroxide, alkali metal carbonate, alkali metal hydrogencarbonate, an organic base such as trialkylamine, and the like.

The reaction temperature is not critical, and the reaction is usually carried out at ambient temperature, under warming or under heating. The present reaction is preferably carried out in the presence of alkali metal halide [e.g., sodium iodide, potassium iodide, etc.], alkali metal thiocyanate [e.g., sodium thiocyanate, potassium thiocyanate, etc.], and the like.

Anion X^{\hat{-}} may be one derived from a leaving group Y, and it may be converted to other anion by a conventional method.

### Process 3-(ii)

The compound [I] or a salt thereof can be prepared by subjecting the compound [VIII] or a salt thereof to elimination reaction of the carboxy protecting group.

Elimination reaction is carried out in similar manner to the reaction in the aforementioned Process 2, and therefore the reagents to be used and reaction conditions (e.g., solvent, reaction temperature, etc.) can be referred to those of Process 2.

### Process 4

The compound [If] or a salt thereof can be prepared by subjecting the compound [Ie] or a salt thereof to elimination reaction of the hydroxy protecting group.

Suitable method of this elimination reaction includes conventional one such as hydrolysis, reduction and the like.

### (i) For Hydrolysis:

The hydrolysis is preferably carried out in the presence of a base or an acid including Lewis acid.

Suitable base includes an inorganic base and an organic base such as an alkali metal [e.g., sodium, potassium, etc.], an alkaline earth metal [e.g., magnesium, calcium, etc.], the hydroxide or carbonate or hydrogencarbonate thereof, trialkylamine [e.g., trimethylamine, triethylamine, etc.], picoline, 1,5-diazabicyclo[4.3.0]non-5-ene, 1,4-diazabicyclo[2.2.2]octane, 1,8-diazabicyclo[5.4.0]undec-7-ene, and the like.

Suitable acid includes an organic acid [e.g., formic acid, acetic acid, propionic acid, trichloroacetic acid, trifluoroacetic acid, etc.], and an inorganic acid [e.g., hydrochloric acid, hydrobromic acid, sulfuric acid, hydrogen chloride, hydrogen bromide, etc.].

The elimination using Lewis acid such as trihaloacetic acid [e.g., trichloroacetic acid, trifluoroacetic acid, etc.] and the like is preferably carried out in the presence of cation trapping agents [e.g., anisole, phenol, etc.].

The reaction is usually carried out in a solvent such as water, alcohol [e.g., methanol, ethanol, etc.], methylene chloride, tetrahydrofuran, a mixture thereof or any other solvent which does not adversely influence the reaction. A liquid base or acid can be also used as a solvent.

The reaction temperature is not critical and the reaction is usually carried out under cooling to warming.

### (ii) For Reduction:

Reduction is carried out in a conventional manner, including chemical reduction and catalytic reduction.

Suitable reducing reagents to be used in chemical reduction are a combination of a metal [e.g., tin, zinc, iron, etc.] or metallic compound [e.g., chromium chloride, chromium acetate, etc.] and an organic acid or inorganic acid [e.g., formic acid, acetic acid, propionic acid, trifluoroacetic acid, p-toluenesulfonic acid, hydrochloric acid, hydrobromic acid, etc.].

Suitable catalysts to be used in catalytic reduction are conventional ones such as platinum catalysts [e.g., platinum plate, spongy platinum, platinum black, colloidal platinum, platinum oxide, platinum wire, etc.], palladium catalysts [e.g., spongy palladium, palladium black, palladium oxide, palladium on carbon, colloidal palladium, palladium on barium sulfate, palladium on barium carbonate, etc.], nickel catalysts [e.g., reduced nickel, nickel oxide, Raney nickel, etc.], cobalt catalysts [e.g., reduced cobalt, Raney cobalt, etc.], iron catalysts [e.g., reduced iron, Raney iron, etc.], copper catalysts [e.g., reduced copper, Raney copper, Ullman copper, etc.] and the like.

The reduction is usually carried out in a conventional solvent which does not adversely influence the reaction such as water, methanol, ethanol, propanol, N,N-dimethylformamide or a mixture thereof.

Additionally, in case that the above-mentioned acids to be used in chemical reduction are liquid, they can also be used as a solvent.

Further, a suitable solvent to be used in catalytic reduction may be the above-mentioned solvent, and other conventional solvent such as diethyl ether, dioxane, tetrahydrofuran, etc., or a mixture thereof.

The reaction temperature of this reduction is not critical and the reaction is usually carried out under cooling to warming.

When R⁵ is protected amino, the amino protecting group in R⁵ can be eliminated by a conventional method such as hydrolysis.

### Process 5

The compound [Ih] or a salt thereof can be prepared by cyclizing R³c of the compound [Ig] or a salt thereof.

This reaction can be carried out in a similar manner to the reaction in Examples 11 and 18 mentioned below.

Processes A and B for the preparation of the starting compounds are explained in detail in the following.

### Process A-(i)

The compound [XII] or a salt thereof can be prepared by reacting the compound [X] or a salt thereof with the compound [XI] or a salt thereof.

This reaction can be carried out in a similar manner to the reaction in the aforementioned Process 3-(i), and therefore the reagents, to be used and reaction conditions (e.g., solvent, reaction temperature, etc.) can be referred to those of Process 3-(i).

### Process A-(ii)

The compound [II] or a salt thereof can be prepared by subjecting the compound [XII] or a salt thereof to elimination reaction of the amino protecting groups in R¹² and R¹⁴ and the carboxy protecting group in R¹³.

This reaction can be carried out in a similar manner to the reaction in the aforementioned Process 2, and therefore the reagents to be used and reaction conditions (e.g., solvent, reaction temperature, etc.) can be referred to those of Process 2.

### Process B

The compound [VI] or a salt thereof can be prepared by reacting the compound [XIII] or its reactive derivative at the amino group, or a salt thereof with the compound [XIV] or its reactive derivative at the carboxy group, or a salt thereof.

This reaction can be carried out in a similar manner to the reaction in the aforementioned Process 1, and therefore the reagents to be used and reaction conditions (e.g., solvent, reaction temperature, etc.) can be referred to those of Process 1.

The compounds obtained by the above Processes 1 to 4 and A and B can be isolated and purified by a conventional method such as pulverization, recrystallization, column chromatography, reprecipitation, and the like.

It is to be noted that the compound [I] may include one or more stereoisomer(s) such as optical isomer(s) and geometrical isomer(s) due to asymmetric carbon atom(s) and double bond(s), and all of such isomers and mixtures thereof are included within the scope of this invention.

The object compounds [I] and a pharmaceutically acceptable salt thereof include solvates [e.g., enclosure compounds (e.g., hydrate, etc.)].

The object compound [I] and a pharmaceutically acceptable salt thereof are novel and exhibit high antimicrobial activity by inhibiting the growth of a wide variety of pathogenic microorganisms including Gram-positive and Gram-negative microorganisms, and are useful as antimicrobial agents.

Now in order to show the utility of the object compound [I], the test data on MIC (minimal inhibitory concentration) of a representative compound of this invention are shown in the following.

### Test Method:

In vitro antibacterial activity was determined by the two-fold agar-plate dilution method as described below.

One loopful of an overnight culture of each test strain in Trypticase-soy broth (10⁶ viable cells per ml) was streaked on heart infusion agar (HI-agar) containing graded concentrations of representative test compound, and the minimal inhibitory concentration (MIC) was expressed in μg/ml after incubation at 37° C. for 20 hours.

### Test Compound

Compound (A): 7β-[(Z)-2-(5-amino-1,2,4-thiadiazol-3-yl)-2-(1-carboxy-1-methylethoxyimino)acetamido]-3-{3-[1-(3-aminopropyl)guanidino]-2,3-dihydro-5-(1H-imidazo[1,2-b]pyrazolio)}methyl-3-cephem-4-carboxylate sulfate (Example 1)
Compound (B): 7β-[(Z)-2-(5-amino-1,2,4-thiadiazol-3-yl)-2-(1-carboxy-1-methylethoxyimino)acetamido]-3-{3-[1-(2-aminoethyl)guanidino]-4,5,6,7-tetrahydro-1-(pyrazolo[1,5-a]pyrimidinio)}methyl-3-cephem-4-carboxylate sulfate (Example 8)

### Test Results:

TABLE 1

| Test strain | Test compound | MIC (μg/ml) |
|---|---|---|
| Pseudomonas aeruginosa | Compound (A) | 0.5 |
| FP 1380* | Compound (B) | 0.5 |
| | Aztreonam | 64 |
| Pseudomonas aeruginosa | Compound (A) | 2 |
| 19136** | Compound (B) | 2 |
| | Aztreonam | 4 |
| Pseudomonas aeruginosa | Compound (A) | 8 |
| 22027** | Compound (B) | 8 |
| | Aztreonam | 32 |

*Pseudomonas aeruginosa FP 1380: AmpC β-lactamase overproduced strain
**Pseudomonas aeruginosa 19136 and 22027: metallo β-lactamase producing strains

For therapeutic administration, the object compound [I] and a pharmaceutically acceptable salt thereof of the present invention are used in the form of a conventional pharmaceutical preparation which contains said compound as an active ingredient, in admixture with a pharmaceutically acceptable carrier such as an organic or inorganic solid, or liquid excipient which is suitable for oral, parenteral or external administration. The pharmaceutical preparations may be in a solid form such as tablet, granule, powder or capsule, or in a liquid form such as solution, suspension, syrup, emulsion or lemonade, or the like.

If needed, there may be included in the above preparations auxiliary substances, stabilizing agents, wetting agents and other commonly used additives such as lactose, citric acid, tartaric acid, stearic acid, magnesium stearate, terra alba, sucrose, corn starch, talc, gelatin, agar, pectin, peanut oil, olive oil, cacao butter, ethylene glycol, and the like.

While the dosage of the compound [I] may vary depending upon the age, conditions of the patient, a kind of diseases, a kind of the compound [I] to be applied, etc., in general amounts between 1 mg and 4,000 mg or even more per day may be administered to a patient. An average single dose of about 50 mg, 100 mg, 250 mg, 500 mg, 1000 mg or 2000 mg of the object compounds [I] of the present invention may be used in treating diseases infected by pathogenic microorganisms.

As used herein, the term “mammal” includes humans and other animals (e.g., mouse, rat, swine, dog, cat, horse, bovine, etc.), preferably humans.

### EXAMPLES

The following Preparations and Examples are given for the purpose of illustrating the present invention in more detail.

The abbreviations in Preparations and Examples mean the following.

THF: tetrahydrofuran

DMF: N,N-dimethylformamide

EtOAc: ethyl acetate

CH₂Cl₂: methylene chloride

MgSO₄: magnesium sulfate

MeOH: methanol

NaHCO₃: sodium hydrogen carbonate

KHSO₄: potassium hydrogen sulfate

IPA: isopropyl alcohol

IPE: isopropyl ether

NaI: sodium iodide

### Preparation 1

To a solution of 3-(tritylamino)propionic acid (99.5 g) and triethylamine (33.4 g) in tetrahydrofuran (1200 ml) was added dropwise methyl chloroformate (31.2 g) under ice-cooling. The mixture was stirred at the same temperature for 40 minutes. To the reaction mixture was added a solution of 2,3-dihydro-1H-imidazo[1,2-b]pyrazol-7-amine sulfate (73.5 g) and triethylamine (66.8 g) in water (600 ml), and the mixture was stirred at room temperature for 30 minutes. To the reaction mixture was added chloroform (1500 ml), and the layers were separated. The organic layer was washed successively with 10% aqueous citric acid solution, brine and saturated aqueous sodium hydrogen carbonate solution. The organic layer was dried over anhydrous magnesium sulfate, filtered and concentrated in vacuo. The residue, was triturated with ethyl acetate to give N-(2,3-dihydro-1H-imidazo[1,2-b]pyrazol-7-yl)-3-(tritylamino)propionamide (60.1 g) as a solid.

¹H-NMR (CDCl₃) δ 2.42-2.44 (2H, m), 2.55-2.58 (2H, m), 3.95-3.98 (2H, m), 4.17-4.20 (2H, m), 4.61 (1H, brs), 7.14 (1H, s), 7.20-7.43 (15H, m), 8.82 (1H, brs)

### Preparation 2

To a solution of N-(2,3-dihydro-1H-imidazo[1,2-b]pyrazol-7-yl)-3-(tritylamino)propionamide (1.31 g) in methylene chloride (25 ml) was added 1.5 mol/l diisobutylaluminum hydride solution in toluene (12.0 ml), and the mixture was refluxed for 6.5 hours under a nitrogen atmosphere. To the reaction mixture were added methylene chloride (30 ml), sodium fluoride (1.5 g) and water (0.6 ml) under ice-cooling. After stirring at room temperature for 15 minutes, di-tert-butyl ({[(trifluoromethyl)sulfonyl]imino}methylene)biscarbamate (2.94 g) was added to the reaction mixture, and the whole mixture was refluxed under a nitrogen atmosphere for 16 hours. After cooling, the insoluble materials were filtered off, and the filtrate was washed successively with water and brine. The organic layer was dried over anhydrous sodium sulfate, filtered and concentrated in vacuo. The residue was purified by column chromatography on silica gel eluting with methylene chloride/methanol (20/1). The eluate containing a desired product was concentrated, and the residue was triturated with a mixed solvent of hexane and diisopropyl ether to give di-tert-butyl ({(2,3-dihydro-1H-imidazo[1,2-b]pyrazol-7-yl)[3-(tritylamino)propyl]amino}methylylidene)biscarbamate (464 mg) as a solid.

¹H-NMR (CDCl₃) δ 1.36 (18H, brs), 1.76 (2H, tt, J=6.6, 6.4 Hz), 2.08 (2H, t, J=6.4 Hz), 3.82 (2H, t, J=6.6 Hz), 3.88 (2H, t, J=6.9 Hz), 4.10 (2H, t, J=7.8 Hz), 7.03 (1H, s), 7.17 (3H, t, J=7.1 Hz), 7.25 (6H, dd, J=7.8, 7.1 Hz), 7.46 (6H, d, J=7.8 Hz), 9.34 (1H, br)

### Example 1

To a solution of 4-methoxybenzyl 7β-[(Z)-2-(5-amino-1,2,4-thiadiazol-3-yl)-2-(1-tert-butoxycarbonyl-1-methylethoxyimino)acetamido]-3-chloromethyl-3-cephem-4-carboxylate (545 mg) in N,N-dimethylformamide (1.6 ml) was added N-(trimethylsilyl)acetamide (525 mg), and the mixture was stirred at room temperature for 35 minutes. To the solution was added potassium iodide (186 mg), and the mixture was stirred at room temperature for 37 minutes. To the reaction mixture was added a solution of di-tert-butyl ({(2,3-dihydro-1H-imidazo[1,2-b]pyrazol-7-yl)[3-(tritylamino)propyl]amino}methylylidene)biscarbamate (533 mg) in N,N-dimethylformamide (1.0 ml), and the whole mixture was stirred at 35° C. to 40° C. for 2 hours and then at 40° C. to 45° C. for 1.5 hours. To the resulting reaction mixture was added ethyl acetate (50 ml), and the solution was washed successively with water (40 ml×2), 10% aqueous sodium trifluoroacetate solution (40 ml) and brine (40 ml), dried over anhydrous sodium sulfate and filtered. The filtrate was concentrated to about 5 g in vacuo. The concentrate was poured into diisopropyl ether (80 ml) and the resulting precipitate was collected by filtration and dried in vacuo. To a solution of the solid in methylene chloride (2.5 ml) were added anisole (0.84 ml) and trifluoroacetic acid (2.5 ml). The resulting solution was stirred at room temperature for 4 hours and poured into diisopropyl ether (80 ml). The resulting precipitate was collected by filtration and dried in vacuo to give a crude product (590 mg), which was purified by preparative high-performance liquid chromatography (HPLC) utilizing an ODS column. The eluate containing a desired product was concentrated to about 20 ml in vacuo. The concentrate was further purified by preparative HPLC utilizing an ODS column eluting with 15% acetonitrile/water. The eluate was concentrated to about 10 ml in vacuo, and 0.05 mol/l sulfuric acid (1.70 ml) was added thereto. The resulting solution was lyophilized to give 70-[(Z)-2-(5-amino-1,2,4-thiadiazol-3-yl)-2-(1-carboxy-1-methylethoxyimino)acetamido]-3-{3-[1-(3-aminopropyl)guanidino]-2,3-dihydro-5-(1H-imidazo[1,2-b]pyrazolio)}methyl-3-cephem-4-carboxylate sulfate (47 mg) as an amorphous solid.

¹H-NMR (D₂O) δ 1.60 (3H, s), d 1.61 (3H, s), 2.02 (2H, quint, J=7.8 Hz), 3.02 (2H, t, J=7.8 Hz), 3.35 (1H, d, J=17.9 Hz), 3.58-3.73 (2H, m), 3.68 (1H, d, J=17.9 Hz), 4.22 (2H, t, J=8.7 Hz), 4.41 (1H, q, J=8.7 Hz), 4.49 (1H, q, J=8.7 Hz), 4.98 (1H, d, J=15.4 Hz), 5.10 (1H, d, J=15.4 Hz), 5.28 (1H, d, J=4.8 Hz), 5.85 (1H, d, J=4.8 Hz), 8.27 (1H, s)

### Preparation 3

To a solution of lithium bis(trimethylsilyl)amide (0.5 M in THF, 400 ml) was added a solution of acetonitrile (8.5 g) in THF (50 ml) dropwise at −70° C. The mixture was stirred for 30 minutes at the same temperature. To the reaction mixture was added 1-methyl-5-(tritylamino)-1H-pyrazole-4-carbaldehyde (20.0 g) and the mixture was stirred for 1 hour at the same temperature, then allowed to warm to room temperature. To the resulting solution was added 10% aqueous potassium hydrogen sulfate solution, and the mixture was extracted with ethyl acetate. The combined layer of ethyl acetate was washed with brine, dried over anhydrous magnesium sulfate, filtered and concentrated in vacuo. The residue was recrystallized from ethyl acetate-diisopropyl ether to give 3-hydroxy-3-[1-methyl-5-(tritylamino)-1H-pyrazol-4-yl]propanenitrile as white crystals (21.9 g).

¹H-NMR (DMSO-d₆) δ 2.02 (1H, dd, J=16.6, 4.0 Hz), 2.21 (1H, dd, J=16.6, 7.2 Hz), 2.75 (3H, s), 4.17-4.25 (1H, m), 5.19 (1H, d, J=4.5 Hz), 5.82 (1H, s), 7.11-7.31 (16H, m)

### Preparation 4

A solution of 3-hydroxy-3-[1-methyl-5-(tritylamino)-1H-pyrazol-4-yl]propanenitrile (9.58 g) and di-tert-butyl dicarbonate (7.67 g) in ethanol (300 ml) was treated with platinum oxide (2.0 g) under a hydrogen atmosphere for 5 days at room temperature. After the catalyst was filtered off, the filtrate was concentrated in vacuo. The residue was purified with silica gel column chromatography to give tert-butyl {3-hydroxy-3-[1-methyl-5-(tritylamino)-1H-pyrazol-4-yl]propyl}carbamate as an amorphous (7.49 g).

¹H-NMR (DMSO-d₆) δ 1.20-1.60 (2H, m), 1.37 (9H, s), 2.55-2.85 (2H, m), 2.76 (3H, s), 3.85-4.00 (1H, m), 4.18 (1H, d, J=4.4 Hz), 5.65 (1H, s), 6.30-6.50 (1H, m), 7.05-7.35 (16H, m)

### Preparation 5

To a solution of tert-butyl {3-hydroxy-3-[1-methyl-5-(tritylamino)-1H-pyrazol-4-yl]propyl}carbamate (5.00 μg) in THF (100 ml) were added diphenylphosphoryl azide (2.52 ml), triphenylphosphine (3.58 g) and diethyl azodicarboxylate (2.38 g) under ice-cooling. This mixture was stirred at room temperature for 2 hours. To the resulting solution was added water and extracted with ethyl acetate, and the organic layer was washed with brine. The extract was dried over anhydrous magnesium sulfate, filtered and concentrated in vacuo. The residue was purified with silica gel column chromatography to give tert-butyl {3-azido-3-[1-methyl-5-(tritylamino)-1H-pyrazol-4-yl]propyl}carbamate (4.0 g) as crystals.

¹H-NMR (DMSO-d₆) δ 1.37 (9H, s), 1.37-1.70 (2H, m), 2.55-2.90 (2H, m), 2.78 (3H, s), 3.95-4.20 (1H, m), 5.95 (1H, s), 6.60-6.75 (1H, m), 7.10-7.50 (16H, m)

### Preparation 6

tert-Butyl {3-azido-3-[1-methyl-5-(tritylamino)-1H-pyrazol-4-yl]propyl}carbamate (4.00 g) in THF (80 ml) was treated with palladium on carbon (2.0 g) under a hydrogen atmosphere for 2 hours at room temperature. After the catalyst was filtered off, the filtrate was concentrated in vacuo. The residue was diluted with THF (60 ml). To this dilution were added di-tert-butyl ({[(trifluoromethyl)sulfonyl]imino}methylene)biscarbamate (7.19 g) and triethylamine (4.2 ml). The mixture was stirred at 50° C. for 48 hours. After cooling, water was added to the mixture, and the mixture was extracted with ethyl acetate. The organic layer was washed with brine, dried over anhydrous sodium sulfate, filtered and concentrated in vacuo. The residue was purified with column chromatography on silica gel to give di-tert-butyl [(Z)-({3-[(tert-butoxycarbonyl)amino]-1-[1-methyl-5-(tritylamino)-1H-pyrazol-4-yl]propyl}amino)methylylidene]biscarbamate (600 mg) as an amorphous solid.

¹H-NMR (DMSO-d₆) δ 1.21 (9H, s), 1.36 (9H, s), 1.45 (9H, s), 1.60-2.00 (2H, m), 2.55-2.75 (2H, m), 3.34 (3H, s), 4.28-4.43 (1H, m), 5.99 (1H, s), 6.45-6.61 (1H, m), 7.10-7.40 (16H, m), 8.17 (1H, d, J=6.9 Hz)

### Example 2

5-Amino-4-(3-amino-1-{[amino(imino)methyl]amino}propyl)-2-{[(6R,7R)-7-({(2Z)-2-(5-amino-1,2,4-thiadiazol-3-yl)-2-[(1-carboxy-1-methylethoxy)imino]acetyl]amino)-2-carboxy-8-oxo-5-thia-1-azabicyclo[4.2.0]oct-2-en-3-yl]methyl}-1-methyl-1H-pyrazol-2-ium hydrogen sulfate (46 mg) was synthesized according to a similar manner to that of Example 1.

IR (KBr) 1772, 1668, 1653, 1525, 1111 cm⁻¹

¹H-NMR (D₂O) δ 1.45-1.60 (2H, m), 1.55 (6H, s), 2.15-2.34 (2H, m), 3.05-3.22 (2H, m), 3.15 and 3.43 (2H, ABq, J=17.3 Hz), 3.70 (3H, s), 4.80-5.20 (2H, m), 5.24 (1H, d, J=4.8 Hz), 5.83 (1H, d, J=4.8 Hz), 7.96 (1H, s)

Mass (m/z) 705.25

### Preparation 7

To a solution of [1-methyl-5-(tritylamino)-1H-pyrazol-4-yl]formamide (10.82 g) in DMF (110 ml) was added sodium hydride (60% dispersion in mineral oil, 828 mg) under ice-cooling, and the mixture was stirred at room temperature for 1 hour. To the reaction mixture were added 3-bromo-N-trityl-1-propanamine (7.88 g) and sodium iodide (3.1 g), and the solution was stirred at room temperature for 3 hours. To this solution was added water, and the mixture was extracted with ethyl acetate. The organic layer was washed with water and brine, dried over anhydrous magnesium sulfate, filtered and concentrated in vacuo. The residue was purified with column chromatography on silica gel to give [1-methyl-5-(tritylamino)-1H-pyrazol-4-yl][3-(tritylamino)propyl]formamide as an amorphous solid (10.82 g).

¹H-NMR (DMSO-d₆) δ 1.30-1.70 (2H, m), 1.70-1.95 (2H, m), 2.70-2.90 (2H, m), 2.75 (3H, s), 6.02 (1H, s), 7.00-7.40 (15H, m), 7.54 (1H, s)

### Preparation 8

To a solution of [1-methyl-5-(tritylamino)-1H-pyrazol-4-yl][3-(tritylamino)propyl]formamide (6.27 g) in methanol (63 ml) was added concentrated hydrochloric acid (7.7 ml), and the mixture was stirred at room temperature for 8 hours. To this mixture were added saturated aqueous sodium hydrogen carbonate solution, and the volatiles were evaporated off. The residue was extracted with ethyl acetate. The organic layer was dried over anhydrous magnesium sulfate, filtered and concentrated in vacuo to give crude 1-methyl-N-4-[3-(tritylamino)propyl]-1H-pyrazole-4,5-diamine as an amorphous solid. This compound was used to the next step without further purification.

### Preparation 9

To the solution of crude 1-methyl-N-4-[3-(tritylamino)propyl]-1H-pyrazole-4,5-diamine synthesized above in THF (75 ml) was added di-tert-butyl ({[(trifluoromethyl)sulfonyl]imino}methylene)biscarbamate (7.19 g), and the mixture was stirred at 50° C. overnight. After cooling, water was added to the mixture, and the mixture was extracted with ethyl acetate. The organic layer was washed with brine, dried over anhydrous sodium sulfate, filtered and concentrated in vacuo. The residue was purified with column chromatography on silica gel to give di-tert-butyl ((E)-{(5-amino-1-methyl-1H-pyrazol-4-yl)[3-(tritylamino)propyl]amino}methylylidene)biscarbamate (2.5 g) as an amorphous solid.

¹H-NMR (DMSO-d₆) δ 1.31 (9H, s), 1.33 (9H, s), 1.55-1.57 (2H, m), 1.80-2.05 (2H, m), 3.45-3.60 (2H, m), 3.50 (3H, s), 5.00 (2H, br-s), 6.89 (1H, s), 7.08-7.45 (16H, m), 8.50-8.80 (1H, br)

### Example 3

5-Amino-4-[[amino(imino)methyl](3-aminopropyl)amino]-2-{[(6R,7R)-7-[((2Z)-2-(5-amino-1,2,4-thiadiazol-3-yl)-2-{[(1S)-1-carboxyethoxy]imino}acetyl)amino]-2-carboxy-8-oxo-5-thia-1-azabicyclo[4.2.0]oct-2-en-3-yl}methyl)-1-methyl-1H-pyrazol-2-ium hydrogen sulfate (15 mg) was synthesized according to a similar manner to that of Example 1.

IR (KBr) 1774, 1662, 1527, 1109 cm¹

¹H-NMR (D₂O) δ 1.53 (3H, d, J=6.9 Hz), 1.90-2.13 (2H, m), 2.80-3.09 (2H, m), 3.30 (1H, d, J=17.7 Hz), 3.40-3.80 (3H, m), 3.76 (3H, s), 4.83-5.20 (3H, m), 5.20-5.35 (1H, m), 5.78-5.90 (1H, m), 8.10-8.30 (1H, m)

Mass (m/z) 679.36

### Example 4

5-Amino-4-[[amino(imino)methyl](3-aminopropyl)amino]-2-{[(6R,7R)-7-({(2Z)-2-(5-amino-1,2,4-thiadiazol-3-yl)-2-[(1-carboxy-1-methylethoxy)imino]acetyl}amino)-2-carboxy-8-oxo-5-thia-1-azabicyclo[4.2.0]oct-2-en-3-yl]methyl}-1-methyl-1H-pyrazol-2-ium hydrogen sulfate as an amorphous solid (55 mg) was synthesized according to a similar manner to that of Example 1.

IR (KBr) 1776, 1655, 1597 cm⁻¹

¹H-NMR (D₂O) δ 1.61 (3H, s), 1.62 (3H, s), 1.95-2.10 (2H, m), 2.95-3.20 (2H, m), 3.33 (1H, d, J=17.9 Hz), 3.60-3.80 (3H, m), 3.76 (3H, s), 4.92-5.03 (1H, m), 5.10-5.23 (1H, m), 5.24-5.32 (1H, m), 5.82-5.90 (1H, m), 8.12-8.27 (1H, m)

Mass (m/z) 611.50

### Example 5

5-Amino-4-[[amino(imino)methyl](3-aminopropyl)amino]-2-[((6R,7R)-7-{[(2Z)-2-(5-amino-1,2,4-thiadiazol-3-yl)-2-(methoxyimino)acetyl]amino}-2-carboxy-8-oxo-5-thia-1-azabicyclo[4.2.0]oct-2-en-3-yl)methyl]-1-methyl-1H-pyrazol-2-ium hydrogen sulfate (132 mg) was synthesized according to a similar manner to that of Example 1.

¹H-NMR (D₂O) δ 1.31-1.47 (2H, m), 3.71 and 3.97 (2H, ABq, J=18.6 Hz), 3.76 (3H, s), 4.10 (3H, s), 4.55-5.05 (6H, m), 5.36 (1H, d, J=4.8 Hz), 6.00 (1H, d, J=4.8 Hz), 7.69 (1H, s)

### Example 6

5-Amino-4-[[amino(imino)methyl](3-aminopropyl)amino]-2-{[(6R,7R)-7-({(2Z)-2-(5-amino-1,2,4-thiadiazol-3-yl)-2-[(carboxymethoxy)imino]acetyl}amino)-2-carboxy-8-oxo-5-thia-1-azabicyclo[4.2.0]oct-2-en-3-yl]methyl}-1-methyl-1H-pyrazol-2-ium hydrogen sulfate (48.5 mg) was synthesized according to a similar manner to that of Example 1.

IR (KBr) 1774, 1660, 1655, 1113 cm⁻¹

¹H-NMR (D₂O) δ 1.90-2.15 (2H, m), 3.03 (2H, t, J=7.8 Hz), 3.31 (1H, d, J=17.7 Hz), 3.45-3.85 (3H, m), 3.75 (3H, m), 4.82-5.14 (2H, m), 4.87 (2H, s), 5.27 (1H, d, J=4.7 Hz), 5.87 (1H, d, J=4.7 Hz), 821 (1H, br-s)

Mass (m/z) 652.48

### Preparation 10

To a solution of 4,5,6,7-tetrahydropyrazolo[1,5-a]pyrimidine (22 g) in sulfuric acid (100 ml) was added potassium nitrate (19.9 g) under ice-cooling, and the reaction mixture was stirred at ambient temperature for 22 hours. The reaction mixture was added to ice-water (300 ml) and stirred for 1 hour. The resultant precipitate was collected by filtration to give 3-nitro-4,5,6,7-tetrahydropyrazolo[1,5-a]pyrimidine (22.82 g).

¹H-NMR δ 1.97-2.08 (4H, m), 3.96-4.02 (4H, m), 7.85 (1H, s), 7.89 (1H, s)

A solution of 3-nitro-4,5,6,7-tetrahydropyrazolo[1,5-a]pyrimidine (1.68 g) in a mixture of sulfuric acid (0.6 ml), acetic acid (100 ml) and water (10 ml) was treated with 10% palladium on carbon (0.5 g) under a hydrogen atmosphere at room temperature for 6 days. After the catalyst was filtered off, the filtrate was concentrated in vacuo. The residue was triturated with ethanol and dried in vacuo to give 3-amino-4,5,6,7-tetrahydropyrazolo[1,5-a]pyrimidine sulfuric acid salt (2.3 g) as a solid.

¹H-NMR (DMSO-d₆) δ 1.97-2.01 (2H, m), 3.22 (2H, t, J=5.0 Hz), 3.98 (2H, t, J=6.0 Hz), 7.22 (1H, s)

### Preparation 11

To a suspension of 3-(tritylamino)propionic acid (13.3 g) in tetrahydrofuran (100 ml) were added triethylamine (5.58 ml) and methyl chloroformate (3.07 ml) under ice-cooling, and the mixture was stirred at room temperature for 25 minutes. To the reaction mixture was added a solution of 4,5,6,7-tetrahydropyrazolo[1,5-a]pyrimidin-3-amine sulfate (7.35 g) and triethylamine (13.9 ml) in water (100 ml), and the mixture was stirred at room temperature for 2.5 hours. To the reaction mixture was added methylene chloride (400 ml), and the layers were separated. The organic layer was washed successively with water, diluted aqueous citric acid solution, 1 mol/l aqueous sodium hydroxide solution and brine. The organic layer was dried over anhydrous sodium sulfate, filtered, and concentrated in vacuo. The residue was triturated with a mixed solvent of methylene chloride and diethyl ether to give N-(4,5,6,7-tetrahydropyrazolo[1,5-a]pyrimidin-3-yl)-3-(tritylamino)propionamide (11.5 g) as a solid.

¹H-NMR (CDCl₃) δ 2.11 (1H, br), 2.12 (2H, quint, J=6.0 Hz), 2.45 (2H, t, J=6.0 Hz), 2.57 (2H, t, J=6.0 Hz), 3.30-3.36 (2H, m), 4.10 (2H, t, J=6.0 Hz), 5.15 (1H, br), 7.11 (1H, s), 7.21 (3H, t, J=7.3 Hz), 7.29 (6H, dd, J=8.3, 7.3 Hz), 7.43 (6H, d, J=8.3 Hz), 8.65 (1H, br)

### Preparation 12

To a solution of N-(4,5,6,7-tetrahydropyrazolo[1,5-a]pyrimidin-3-yl)-3-(tritylamino)propionamide (9.03 mg) in methylene chloride (15 ml) was added 1.5 mol/l diisobutylaluminum hydride solution in toluene (8.00 ml), and the mixture was refluxed for 5.5 hours under a nitrogen atmosphere. To the reaction mixture were added methylene chloride (20 ml), sodium fluoride (1 g) and water (0.4 ml) under ice-cooling. After stirring at room temperature for 15 minutes, di-tert-butyl ({[(trifluoromethyl)sulfonyl]imino}methylene)biscarbamate (1.96 g) was added to the reaction mixture, and the whole mixture was refluxed under a nitrogen atmosphere for 17 hours. After cooling, the insoluble materials were filtered off. The filtrate was diluted with ethyl acetate (200 ml), and the solution was washed successively with water and brine. The organic layer was dried over anhydrous sodium sulfate, filtered and concentrated in vacuo. The residue was purified by column chromatography on silica gel eluting with methylene chloride/methanol (20/1). The eluate containing a desired product was concentrated, and the residue was triturated with a mixed solvent of hexane and diisopropyl ether to give di-tert-butyl ({(4,5,6,7-tetrahydropyrazolo[1,5-a]pyrimidin-3-yl)[3-(tritylamino)propyl]amino}methylylidene)biscarbamate (519 mg) as a solid.

¹H-NMR (CDCl₃) δ 1.35 (18H, brs), 1.50-1.59 (2H, m), 1.66-1.75 (2H, m), 2.07 (2H, t, J=6.2 Hz), 3.14-3.22 (2H, m), 3.72-3.83 (2H, m), 4.02 (2H, t, J=6.2 Hz), 4.36 (1H, br), 6.97 (1H, s), 7.16 (3H, t, J=7.3 Hz), 7.25 (6H, dd, J=7.8, 7.3 Hz), 7.45 (6H, d, J=7.8 Hz), 8.80 (1H, br).

### Example 7

To a solution of 4-methoxybenzyl 7β-[(Z)-2-(5-amino-1,2,4-thiadiazol-3-yl)-2-(1-tert-butoxycarbonyl-1-methylethoxyimino)acetamido]-3-chloromethyl-3-cephem-4-carboxylate (500 mg) in N,N-dimethylformamide (1.5 ml) was added N-(trimethylsilyl)acetamide (482 mg) and the mixture was stirred at room temperature for 36 minutes. To the solution was added potassium iodide (171 mg), and the mixture was stirred at room temperature for 37 minutes. To the reaction mixture was added a solution of di-tert-butyl ({(4,5,6,7-tetrahydropyrazolo[1,5-a]pyrimidin-3-yl)[3-(tritylamino)propyl]amino}methylylidene)biscarbamate (499 mg) in N,N-dimethylformamide (2.0 ml), and the whole mixture was stirred at 35° C. to 40° C. for 2 hours and then at 40° C. to 45° C. for 1.5 hours. To the resulting reaction mixture was added ethyl acetate (50 ml), and the solution was washed successively with water (40 ml×2), 10% aqueous sodium trifluoroacetate solution (40 ml) and brine (40 ml), dried over anhydrous sodium sulfate and filtered. The filtrate was concentrated to about 5 g in vacuo. The concentrate was poured into isopropyl ether (80 ml) and the resulting precipitate was collected by filtration and dried in vacuo. To a solution of the solid in methylene chloride (2.3 ml) were added anisole (0.75 ml) and trifluoroacetic acid (2.3 ml). The resulting solution was stirred at room temperature for 4 hours and poured into diisopropyl ether (80 ml). The resulting precipitate was collected by filtration and dried in vacuo to give a crude product (540 mg), which was purified by preparative high-performance liquid chromatography (HPLC) utilizing an ODS column. The eluate containing a desired product was concentrated to about 20 ml in vacuo. The concentrate was further purified by preparative HPLC utilizing an ODS column eluting with 10% acetonitrile/water. The eluate was concentrated to about 10 ml in vacuo and 0.05 mol/l sulfuric acid (3.49 ml) was added. The resulting solution was lyophilized to give 7β-[(Z)-2-(5-amino-1,2,4-thiadiazol-3-yl)-2-(1-carboxy-1-methylethoxyimino)acetamido]-3-{3-[1-(3-aminopropyl)guanidino]-4,5,6,7-tetrahydro-1-(pyrazolo[1,5-a]pyrimidinio)}methyl-3-cephem-4-carboxylate sulfate (133 mg) as an amorphous solid.

¹H-NMR (D₂O) δ 1.61 (6H, s), 1.93-2.09 (2H, m), 2.09-2.25 (2H, m), 3.01 (2H, t, J=7.6 Hz), 3.33 (1H, d, J=17.9 Hz), 3.40 (2H, t, J=5.3 Hz), 3.45-3.83 (3H, m), 4.07-4.26 (2H, m), 4.91 (1H, d, J=13.8 Hz), 5.05-5.31 (1H, m), 5.14 (1H, brs), 5.85 (1H, brs), 8.06-8.21 (1H, m)

### Preparation 13

To a solution of (tritylamino)acetic acid (79.3 g) and triethylamine (27.8 g) in tetrahydrofuran (1000 ml) was added dropwise methyl chloroformate (26.0 g) under ice-cooling. The mixture was stirred at the same temperature for 30 minutes. To the reaction mixture was added a solution of 4,5,6,7-tetrahydropyrazolo[1,5-a]pyrimidin-3-amine sulfate (59.0 g) and triethylamine (50.6 g) in water (500 ml), and the mixture was stirred at room temperature for 30 minutes. To the reaction mixture was added chloroform (1500 ml), and the layers were separated. The organic layer was washed successively with 10% aqueous citric acid solution, brine and saturated aqueous sodium hydrogen carbonate solution. The organic layer was dried over anhydrous magnesium sulfate, filtered, and concentrated in vacuo. The residue was triturated with ethyl acetate to give N-(4,5,6,7-tetrahydropyrazolo[1,5-a]pyrimidin-3-yl)-2-(tritylamino)acetamide (46.4 g) as a solid.

¹H-NMR (CDCl₃) δ 2.10-2.14 (2H, m), 2.53 (1H, brs), 3.09 (2H, brs), 3.31-3.33 (2H, m), 4.08-4.11 (2H, m), 5.07 (1H, brs), 7.16 (1H, s), 7.19-7.40 (15H, m), 8.67 (1H, brs)

### Preparation 14

To a solution of N-(4,5,6,7-tetrahydropyrazolo[1,5-a]pyrimidin-3-yl)-2-(tritylamino)acetamide (4.38 g) in methylene chloride (80 ml) was added 1.5 mol/l diisobutylaluminum hydride solution in toluene (40.0 ml), and under a nitrogen atmosphere the mixture was refluxed for 8.5 hours. To the reaction mixture were added methylene chloride (80 ml), sodium fluoride (5 g), and then water (2 ml) under ice-cooling. After stirring at room temperature for 15 minutes, di-tert-butyl ({[(trifluoromethyl)sulfonyl]imino}methylene)biscarbamate (9.78 g) was added to the reaction mixture, and the whole mixture was refluxed under a nitrogen atmosphere for 19.5 hours. After cooling, the insoluble materials were filtered off, and the filtrate was washed successively with water and brine. The organic layer was dried over anhydrous sodium sulfate, filtered, and concentrated in vacuo. The residue was triturated with a mixed solvent of methylene chloride and diethyl ether to give di-tert-butyl ({(4,5,6,7-tetrahydropyrazolo[1,5-a]pyrimidin-3-yl)[2-(tritylamino)ethyl]amino}methylylidene)biscarbamate (1.67 g) as a solid.

¹H-NMR (CDCl₃) δ 1.36 (9H, s), 1.43 (9H, s), 1.94-2.03 (2H, m), 2.38-2.53 (2H, m), 2.97-3.10 (2H, m), 3.30 (1H, br), 3.61-3.90 (2H, m), 3.98 (2H, t, J=6.2 Hz), 5.44 (1H, br), 6.98 (1H, s), 7.18 (3H, t, J=7.3 Hz), 7.27 (6H, dd, J=7.8, 7.3 Hz), 7.40 (6H, d, J=7.8 Hz), 8.81 (1H, br)

### Example 8

To a solution of 4-methoxybenzyl 70-[(Z)-2-(5-amino-1,2,4-thiadiazol-3-yl)-2-(1-tert-butoxycarbonyl-1-methylethoxyimino)acetamido]-3-chloromethyl-3-cephem-4-carboxylate (817 mg) in N,N-dimethylformamide (2.0 ml) was added N-(trimethylsilyl)acetamide (788 mg), and the mixture was stirred at room temperature for 30 minutes. To the solution was added potassium iodide (282 mg), and the mixture was stirred at room temperature for 47 minutes. To the reaction mixture was added a solution of di-tert-butyl ({(4,5,6,7-tetrahydropyrazolo[1,5-a]pyrimidin-3-yl)[2-(tritylamino)ethyl]amino}methylylidene)biscarbamate (666 mg) in N,N-dimethylformamide (2.0 ml), and the whole mixture was stirred at 40° C. to 50° C. for 4 hours. To the resulting reaction mixture was added ethyl acetate (50 ml), and the solution was washed successively with water (50 ml×2), 10% aqueous sodium trifluoroacetate solution (50 ml) and brine (40 ml), dried over anhydrous sodium sulfate and filtered. The filtrate was concentrated to about 5 g in vacuo. The concentrate was poured into diisopropyl ether (100 ml), and the resulting precipitate was collected by filtration and dried in vacuo. The obtained solid was dissolved in methylene chloride (3.6 ml), and anisole (1.2 ml) and trifluoroacetic acid (3.6 ml) were added to the solution. The resulting solution was stirred at room temperature for 4 hours and poured into diisopropyl ether (100 ml). The resulting precipitate was collected by filtration and dried in vacuo to give a crude product (855 mg), which was purified by preparative high-performance liquid chromatography (HPLC) utilizing an ODS column. The eluate containing a desired product was concentrated to about 20 ml in vacuo. The concentrate was further purified by preparative HPLC utilizing an ODS column eluting with 15% acetonitrile/water. The eluate was concentrated to about 10 ml in vacuo, and 0.05 mol/l sulfuric acid (2.64 ml) was added thereto. The resulting solution was lyophilized to give 7β-[(Z)-2-(5-amino-1,2,4-thiadiazol-3-yl)-2-(1-carboxy-1-methylethoxyimino)acetamido]-3-{3-[1-(2-aminoethyl)guanidino]-4,5,6,7-tetrahydro-1-(pyrazolo[1,5-a]pyrimidinio)}methyl-3-cephem-4-carboxylate sulfate (139 mg) as an amorphous solid.

¹H-NMR (D₂O) δ 1.61 (3H, s), 1.62 (3H, s), 2.10-2.25 (2H, m), 3.20-3.33 (2H, m), 3.33 (1H, d, J=17.9 Hz), 3.41 (2H, t, J=5.5 Hz), 3.53-4.09 (3H, m), 4.10-4.26 (2H, m), 4.93 (1H, d, J=15.6 Hz), 5.06-5.30 (1H, m), 5.27 (1H, d, J=4.1 Hz), 5.85 (1H, d, J=4.1 Hz), 8.07-8.23 (1H, m)

### Preparation 15

To a solution of 0.5 mol/l lithium bis(trimethylsilyl)amide in tetrahydrofuran (70 ml) was added dropwise a solution of acetonitrile (1.56 g) in tetrahydrofuran (8 ml) under a nitrogen atmosphere at −70° C., and the reaction mixture was stirred at the same temperature for 30 minutes. To the reaction mixture was added 2,3-dihydro-1H-imidazo[1,2-b]pyrazole-7-carbaldehyde (1.37 g), and the reaction mixture was stirred at same temperature for 1 hour. To the reaction mixture was added water (20 ml), and the mixture was washed with chloroform. The aqueous layer was adjusted to about pH 7 by addition of 10% aqueous citric acid solution and chromatographed on Diaion® HP-20 (Mitsubishi Chemical Corporation) eluting with 50% aqueous methanol. The eluate was concentrated in vacuo. The residue was triturated with acetone to give 3-(2,3-dihydro-1H-imidazo[1,2-b]pyrazol-7-yl)-3-hydroxypropionitrile (1.17 g) as a solid.

¹H-NMR (DMSO-d₆) δ 2.74-2.83 (2H, m), 3.80-3.84 (2H, m), 3.98-4.01 (2H, m), 4.65-4.69 (1H, m), 4.45 (1H, d, J=4 Hz), 5.62 (1H, brs), 7.18 (1H, s)

### Preparation 16

To acetic anhydride (16 ml) was added formic acid (8 ml), and the mixture was stirred at room temperature for 1 hour. To the resulting mixture was added 3-(2,3-dihydro-1H-imidazo[1,2-b]pyrazol-7-yl)-3-hydroxypropionitrile (1.78 g), and the reaction mixture was stirred at room temperature for 2 hours. After evaporation of the solvent in vacuo, and the residue was triturated with acetone to give N-(2,3-dihydro-1-formyl-1H-imidazo[1,2-b]pyrazol-7-yl)-3-hydroxypropionitrile (1.46 g) as a solid.

¹H-NMR (DMSO-d₆) δ 2.72-2.85 (0.6H, m), 2.90-2.92 (1.4H, m), 4.27-4.32 (1.4H, m), 4.35-4.38 (2H, m), 4.55-4.58 (0.6H, m), 4.97-5.00 (0.7H, m), 5.39-5.41 (0.3H, m), 5.63 (0.3H, d, J=5.5 Hz), 6.03 (0.7H, d, J=4.1 Hz), 7.38 (0.7H, s), 7.46 (0.3H, s), 8.22 (0.3H, s), 9.00 (0.7H, s)

### Preparation 17

A suspension of N-(2,3-dihydro-1-formyl-1H-imidazo[1,2-b]pyrazol-7-yl)-3-hydroxypropionitrile (620 mg) and di-tert-butyl dicarbonate (980 mg) in methanol (30 ml) was treated with platinum(IV) oxide (150 mg) under a hydrogen atmosphere at room temperature for 4 days. After the catalyst was filtered off, the filtrate was concentrated in vacuo. The residue was purified by column chromatography on silica gel eluting with 7% methanol/chloroform to give tert-butyl 3-(2,3-dihydro-1-formyl-1H-imidazo[1,2-b]pyrazol-7-yl)-3-hydroxypropylcarbamate (320 mg) as a solid.

¹H-NMR (CDCl₃) δ 1.44 (2.7H, s), 1.46 (6.3H, s), 1.86-1.88 (1.4H, m), 1.99-2.00 (0.6H, m), 3.14-3.17 (0.7H, m), 3.22-3.45 (0.3H, m), 3.59-3.66 (0.3H, m), 3.69-3.75 (0.7H, m), 4.32-4.35 (1.4H, m), 4.38-4.41 (0.6H, m), 4.46-4.50 (1.4H, m), 4.56-4.59 (0.6H, m), 4.71-4.73 (0.7H, m), 4.86 (0.7H, brs), 4.89 (0.3H, br), 5.11 (0.3H, br), 7.30 (0.7H, s), 7.40 (0.3H, s), 8.20 (0.3H, s), 9.15 (0.7H, s)

### Preparation 18

To a solution of tert-butyl 3-(2,3-dihydro-1-formyl-1H-imidazo[1,2-b]pyrazol-7-yl)-3-hydroxypropylcarbamate (3.1 g), diphenylphosphoryl azide (3.3 g) and triphenylphosphine (3.7 g) in tetrahydrofuran (100 ml) was added diisopropyl azodicarboxylate (2.8 ml), and the mixture was stirred at room temperature for 3 hours. The solvent was evaporated in vacuo to give crude tert-butyl 3-azido-3-(2,3-dihydro-1-formyl-1H-imidazo[1,2-b]pyrazol-7-yl)propylcarbamate (2.3 g) as an oil. This product was used directly in the next step without further purification. A solution of the above oily product [i.e., tert-butyl 3-amino-3-(2,3-dihydro-1-formyl-1H-imidazo[1,2-b]pyrazol-7-yl)propylcarbamate] in ethanol (100 ml) was treated with 10% palladium on carbon (1.0 g) under a hydrogen atmosphere at room temperature for 71 hours. After the catalyst was filtered off, the filtrate was concentrated in vacuo.

The residue was dissolved in 10% aqueous citric acid solution, and the solution was washed with ethyl acetate. The aqueous layer was adjusted to about pH 10 with 10% aqueous sodium hydroxide solution. The mixture was stirred at room temperature for 1 hour. The reaction mixture was extracted with chloroform. The organic layer was dried over anhydrous magnesium sulfate, filtered and concentrated in vacuo to give tert-butyl 3-amino-3-(2,3-dihydro-1H-imidazo[1,2-b]pyrazol-7-yl)propylcarbamate (2.86 g) as an oil.

¹H-NMR (CDCl₃) δ 1.44 (9H, s), 1.58 (2H, br), 1.71-1.82 (2H, m), 3.11-3.23 (2H, m), 3.87 (1H, t, J=6.0 Hz), 3.97-4.01 (2H, m), 4.10-4.17 (2H, m), 4.40 (1H, br), 4.83 (1H, br), 7.27 (1H, s)

### Preparation 19

To a solution of tert-butyl 3-amino-3-(2,3-dihydro-1H-imidazo[1,2-b]pyrazol-7-yl)propylcarbamate (2.44 g) and triethylamine (1.52 g) in chloroform (50 ml) was added di-tert-butyl ({[(trifluoromethyl)sulfonyl]imino}methylene)biscarbamate (5.10 g), and the whole mixture was stirred at room temperature for 16 hours. The reaction mixture was washed successively with 10% aqueous citric acid solution, brine and saturated aqueous sodium hydrogen carbonate solution. The organic layer was dried over anhydrous magnesium sulfate, filtered and concentrated in vacuo. The residue was purified by column chromatography on silica gel eluting with 4% methanol/chloroform to give tert-butyl 3-[2,3-bis(tert-butoxycarbonyl)guanidino]-3-(2,3-dihydro-1H-imidazo[1,2-b]pyrazol-7-yl)propylcarbamate (500 mg) as an oil.

¹H-NMR (CDCl₃) δ 1.44 (9H, s), 1.47 (9H, s), 1.49 (9H, s), 2.08-2.10 (2H, m), 3.15-3.20 (2H, m), 3.92-3.97 (2H, m), 4.08-4.13 (1H, m), 4.20-4.24 (1H, m), 4.72-4.74 (1H, m), 4.90 (1H, brs), 5.41 (1H, brs), 7.27 (1H, s), 8.46 (1H, d, J=6 Hz), 11.19 (1H, brs)

### Example 9

To a solution of 4-methoxybenzyl 7β-[(Z)-2-(5-amino-1,2,4-thiadiazol-3-yl)-2-(1-tert-butoxycarbonyl-1-methylethoxyimino)acetamido]-3-chloromethyl-3-cephem-4-carboxylate (7.30 g) in N,N-dimethylformamide (22 ml) was added N-(trimethylsilyl)acetamide (7.03 g), and the mixture was stirred at room temperature for 30 minutes. To the solution was added potassium iodide (2.49 g) and the mixture was stirred at room temperature for 30 minutes. To the reaction mixture was added tert-butyl 3-[2,3-bis(tert-butoxycarbonyl)guanidino]-3-(2,3-dihydro-1H-imidazo[1,2-b]pyrazol-7-yl)propylcarbamate (5.10 g), and the whole mixture was stirred at 40° C. for 3.5 hours. To the resulting reaction mixture was added ethyl acetate (800 ml) and the solution was washed successively with brine (400 ml), 10% aqueous sodium trifluoroacetate solution (400 ml) and brine (400 ml), dried over anhydrous magnesium sulfate and filtered. The filtrate was concentrated to about 50 ml in vacuo. The concentrate was poured into diisopropyl ether (700 ml), and the resulting precipitate was collected by filtration and dried in vacuo. To a solution of the solid in methylene chloride (30 ml) were added anisole (10 ml) and trifluoroacetic acid (30 ml). The resulting solution was stirred at room temperature for 4 hours and poured into diisopropyl ether (700 ml). The resulting precipitate was collected by filtration and dried in vacuo to give a crude product (8.20 g), which was purified by preparative high-performance liquid chromatography (HPLC) utilizing an ODS column.

The first eluate containing a desired product was concentrated to about 20 ml in vacuo. The concentrate was further purified by preparative HPLC utilizing an ODS column eluting with 15% acetonitrile/water. The eluate was concentrated to about 10 ml in vacuo, and 0.05 mol/l sulfuric acid (8.80 ml) was added thereto. The resulting solution was lyophilized to give 70-[(Z)-2-(5-amino-1,2,4-thiadiazol-3-yl)-2-(1-carboxy-1-methylethoxyimino)acetamido]-3-[7-(3-amino-1-guanidinopropyl)-2,3-dihydro-5-(1H-imidazo[1,2-b]pyrazolio)]methyl-3-cephem-4-carboxylate sulfate (438.2 mg) as an amorphous solid.

The second eluate was worked up as described above (0.05 mol/l sulfuric acid: 8.50 ml) to give 7β-[(Z)-2-(5-amino-1,2,4-thiadiazol-3-yl)-2-(1-carboxy-1-methylethoxyimino)acetamido]-3-[7-(3-amino-1-guanidinopropyl)-2,3-dihydro-5-(1H-imidazo[1,2-b]pyrazolio)]methyl-3-cephem-4-carboxylate sulfate (428.7 mg) as an amorphous solid.

¹H-NMR (D₂O) δ 1.16 (6H, d, J=1.8 Hz), 2.23 (2H, dt, J=7.8 Hz), 3.04-3.17 (2H, m), 3.28 (1H, d, J=17.9 Hz), 3.55 (1H, d, J=17.9 Hz), 4.17 (2H, t, J=8.7 Hz), 4.29-4.41 (2H, m), 4.66 (1H, t, J=7.8 Hz), 4.93 (1H, d, J=15.6 Hz), 5.09 (1H, d, J=15.6 Hz), 5.26 (1H, d, J=5.0 Hz), 5.85 (H, d, J=5.0 Hz), 8.04 (1H, s)

¹H-NMR (D₂O) δ 1.16 (6H, d, J=1.8 Hz), 2.23 (2H, dt, J=7.8 Hz), 3.04-3.17 (2H, m), 3.29 (1H, d, J=17.9 Hz), 3.55 (1H, d, J=17.9 Hz), 4.17 (2H, t, J=8.7 Hz), 4.30 (1H, dt, J=8.7 Hz), 4.39 (1H, dt, J=8.7 Hz), 4.67 (1H, t, J=7.8 Hz), 4.98 (1H, d, J=15.6 Hz), 5.10 (1H, d, J=15.6 Hz), 5.26 (1H, d, J=4.6 Hz), 5.86 (1H, d; J=4.6 Hz), 8.06 (1H, s)

### Preparation 20

To a solution of N-(2,3-dihydro-1H-imidazo[1,2-b]pyrazol-7-yl)-2-(tritylamino)acetamide (2.54 g) in methylene chloride (45 ml) was added 1.5 M diisobutylaluminum hydride solution in toluene (16.0 ml), and the mixture was refluxed under a nitrogen atmosphere. After 4 hours, additional 1.5 M diisobutylaluminum hydride solution in toluene (8.0 ml) was added to the reaction mixture, and the mixture was refluxed for 1.5 hours under a nitrogen atmosphere. To the reaction mixture were added methylene chloride (60 ml), sodium fluoride (3.6 g), and then water (1.2 ml) under ice-cooling. After stirring at room temperature for 15 minutes, di-tert-butyl ({[(trifluoromethyl)sulfonyl]imino}methylene)biscarbamate (3.52 g) was added to the reaction mixture, and the whole mixture was refluxed under a nitrogen atmosphere for 17.5 hours. After cooling, the insoluble materials were filtered off, and the filtrate was concentrated in vacuo. The residue was dissolved in diethyl ether, and the solution was washed successively with water and brine. The organic layer was dried over anhydrous sodium sulfate, filtered and concentrated in vacuo. The residue was purified by column chromatography on silica gel eluting with 3% methanol/methylene chloride. The eluate containing a desired product was concentrated in vacuo to give di-tert-butyl ({(2,3-dihydro-1H-imidazo[1,2-b]pyrazol-7-yl)[2-(tritylamino)ethyl]amino}methylylidene)biscarbamate (990 mg) as an amorphous solid.

¹H-NMR (CDCl₃) δ 1.41 (18H, s), 2.45 (2H, t, J=5.5 Hz), 3.78 (2H, t, J=5.5 Hz), 3.82 (2H, t, J=8.0 Hz), 4.08 (2H, t, J=8.0 Hz), 7.00 (1H, s), 7.18 (3H, t, J=7.3 Hz), 7.27 (6H, dd, J=8.2, 7.3 Hz), 7.43 (6H, d, J=8.2 Hz)

### Example 10

To a solution of 4-methoxybenzyl 7β-[(Z)-2-(5-amino-1,2,4-thiadiazol-3-yl)-2-(1-tert-butoxycarbonyl-1-methylethoxyimino)acetamido]-3-chloromethyl-3-cephem-4-carboxylate (841 mg) in N,N-dimethylformamide (2.0 ml) was added N-(trimethylsilyl)acetamide (810 mg), and the mixture was stirred at room temperature for 30 minutes. To the solution was added potassium iodide (284 mg), and the mixture was stirred at room temperature for 40 minutes. To the reaction mixture was added a solution of di-tert-butyl ({(2,3-dihydro-1H-imidazo[1,2-b]pyrazol-7-yl)[2-(tritylamino)ethyl]amino}methylylidene)biscarbamate (619 mg) in N,N-dimethylformamide (2.0 ml), and the whole mixture was stirred at 40° C. to 45° C. for 4.5 hours. To the resulting reaction mixture was added ethyl acetate (50 ml), and the solution was washed successively with water (50 ml×2), 10% aqueous sodium trifluoroacetate solution (50 ml) and brine (50 ml), dried over anhydrous sodium sulfate and filtered. The filtrate was concentrated to about 6 g in vacuo. The concentrate was poured into diisopropyl ether (100 ml) and the resulting precipitate was collected by filtration and dried in vacuo. To a solution of the solid in methylene chloride (3.9 ml) were added anisole (1.3 ml) and trifluoroacetic acid (3.9 ml). The resulting solution was stirred at room temperature for 4 hours and poured into diisopropyl ether (100 ml). The resulting precipitate was collected by filtration and dried in vacuo to give a crude product (1.02 g), which was purified by preparative high-performance liquid chromatography (HPLC) utilizing an ODS column. The eluate containing a desired product was concentrated to about 20 ml in vacuo. The concentrate was further purified by preparative HPLC utilizing an ODS column eluting with 15% acetonitrile/water. The eluate was concentrated to about 10 ml in vacuo, and 0.05 M sulfuric acid (1.41 ml) was added thereto. The resulting solution was lyophilized to give 7β-[(Z)-2-(5-amino-1,2,4-thiadiazol-3-yl)-2-(1-carboxy-1-methylethoxyimino)acetamido]-3-{3-[1-(2-aminoethyl)guanidino]-2,3-dihydro-5-(1H-imidazo[1,2-b]pyrazolio)}methyl-3-cephem-4-carboxylate sulfate (62 mg) as an amorphous solid.

¹H-NMR (D₂O) δ 1.61 (3H, s), 1.61 (3H, s), 3.29 (2H, t, J=7.6 Hz), 3.36 (1H, d, J=18.1 Hz), 3.69 (1H, d, J=18.1 Hz), 3.92 (2H, t, J=7.6 Hz), 4.24 (2H, t, J=8.7 Hz), 4.42 (1H, q, J=8.7 Hz), 4.50 (1H, q, J=8.7 Hz), 5.00 (1H, d, J=15.4 Hz), 5.10 (1H, d, J=15.4 Hz), 5.29 (1H, d, J=4.6 Hz), 5.86 (1H, d, J=4.6 Hz), 8.30 (1H, s)

### Example 11

A solution of 7-[[amino(imino)methyl](3-aminopropyl)amino]-5-{[(6R,7R)-7-({(2Z)-2-(5-amino-1,2,4-thiadiazol-3-yl)-2-[(1-carboxy-1-methylethoxy)imino]acetyl}amino)-2-carboxy-8-oxo-5-thia-1-azabicyclo[4.2.0]oct-2-en-3-yl]methyl}-2,3-dihydro-1H-imidazo[1,2-b]pyrazol-5-ium hydrogen sulfate (300 mg) in pH 7 phosphate buffer (60 ml) was stirred at 35° C. to 40° C. for 24 hours. The solution was purified by preparative high-performance liquid chromatography (HPLC) utilizing an ODS column. The eluate containing a desired product was concentrated to about 30 ml in vacuo. The concentrate was adjusted to about pH 1 by addition of concentrated hydrochloric acid and chromatographed on Diaion® HP-20 (Mitsubishi Chemical Corporation) eluting with 15% aqueous 2-propanol. The eluate was concentrated to about 30 ml in vacuo, and 2 M aqueous sulfuric acid solution (0.04 ml) was added thereto. The solution was lyophilized to give 5-{[(6R,7R)-7-({(2Z)-2-(5-amino-1,2,4-thiadiazol-3-yl)-2-[(1-carboxy-1-methylethoxy)imino]acetyl}amino)-2-carboxy-8-oxo-5-thia-1-azabicyclo[4.2.0]oct-2-en-3-yl]methyl}-7-(2-iminotetrahydro-1(2H)-pyrimidinyl)-2,3-dihydro-1H-imidazo[1,2-b]pyrazol-5-ium hydrogen sulfate (30 mg).

¹H-NMR δ 1.61 (6H, s), 1.90-2.25 (2H, m), 3.25-3.43 (3H, m), 3.50-3.75 (3H, m), 4.10-4.35 (2H, m), 4.35-4.60 (2H, m), 4.85-5.20 (2H, m), 5.28 (1H, d, J=4.9 Hz), 5.86 (1H, d, J=4.9 Hz), 8.23 (1H, s)

MASS (ESI) 675.3 (M+H⁺)

### Preparation 21

To a suspension of sodium hydride (55% dispersion in mineral oil, 5.5 g) in tetrahydrofuran (460 ml) was added 4,5,6,7-tetrahydropyrazolo[1,5-a]pyrimidine-3-carbaldehyde (17.4 g) under ice-cooling. The mixture was stirred at the same temperature for 30 minutes. To the reaction mixture was added di-tert-butyl dicarbonate (25.0 g), and the mixture was stirred at room temperature for 1 hour. To the reaction mixture was added tetrahydrofuran (300 ml), and the mixture was stirred at the same temperature for 3 hours. To the reaction mixture were added water (300 ml) and ethyl acetate (1000 ml), and the layers were separated. The organic layer was washed successively with water (500 ml×2) and brine (500 ml). The organic layer was dried over anhydrous sodium sulfate, filtered and concentrated in vacuo. The residue was triturated with hexane to give tert-butyl 3-formyl-4,5,6,7-tetrahydropyrazolo[1,5-a]pyrimidine-4-carboxylate (23.5 g) as a solid.

¹H-NMR (CDCl₃) δ 1.51 (9H, s), 2.17-2.23 (2H, m), 3.87-3.91 (2H, m), 4.22 (2H, t, J=6.0 Hz), 7.89 (1H, s), 9.90 (1H, s)

### Preparation 22

To a solution of 0.5 mol/l lithium bis(trimethylsilyl)amide in tetrahydrofuran (400 ml) was added dropwise a solution of acetonitrile (10.9 ml) in tetrahydrofuran (40 ml) under a nitrogen atmosphere at −70° C., and the reaction mixture was stirred at the same temperature for 30 minutes. To the reaction mixture was added tert-butyl 3-formyl-4,5,6,7-tetrahydropyrazolo[1,5-a]pyrimidine-4-carboxylate (20.1 g), and the reaction mixture was stirred at the same temperature for 40 minutes. To the reaction mixture were added water (200 ml) and ethyl acetate (200 ml), and the layers were separated. The organic layer was washed successively with 10% aqueous citric acid solution (200 ml), water (200 ml×2) and brine (200 ml). The organic layer was dried over anhydrous sodium sulfate, filtered and concentrated in vacuo. The residue was triturated with diisopropyl ether to give tert-butyl 3-(2-cyano-1-hydroxyethyl)-4,5,6,7-tetrahydropyrazolo[1,5-a]pyrimidine-4-carboxylate (15.9 g) as a solid.

¹H-NMR (CDCl₃) δ 1.55 (9H, s), 2.09-2.23 (2H, m), 2.80-2.95 (2H, m), 3.61-3.68 (2H, m), 3.93-4.00 (1H, m), 4.14-4.27 (2H, m), 4.37 (1H, brs), 4.89-4.96 (1H, m), 7.52 (1H, s)

### Preparation 23

To a solution of tert-butyl 3-(2-cyano-1-hydroxyethyl)-4,5,6,7-tetrahydropyrazolo[1,5-a]pyrimidine-4-carboxylate (14.6 g) in methanol (500 ml) were added di-tert-butyl dicarbonate (16.4 g) and platinum(IV) oxide (5.0 g), and the mixture was stirred under a hydrogen atmosphere at room temperature for 5 days. After the catalyst was filtered off, the filtrate was concentrated in vacuo, and the residue was purified by column chromatography on silica gel eluting with 10% methanol/chloroform to give tert-butyl 3-[3-(butoxycarbonylamino)-1-hydroxypropyl]-4,5,6,7-tetrahydropyrazolo[1,5-a]pyrimidine-4-carboxylate (16.6 g) as a solid.

¹H-NMR (CDCl₃) δ 1.43 (9H, s), 1.53 (9H, s), 1.84-1.93 (2H, m), 1.98-2.07 (1H, m), 2.07-2.20 (1H, m), 3.11-3.21 (1H, m), 3.36-3.47 (1H, m), 3.52-3.61 (1H, m), 3.93-4.02 (1H, m), 4.10-4.25 (2H, m), 4.56-4.65 (1H, m), 5.14 (1H, brs), 7.46 (1H, s)

### Preparation 24

To a solution of tert-butyl 3-[(3-butoxycarbonylamino)-1-hydroxypropyl]-4,5,6,7-tetrahydropyrazolo[1,5-a]pyrimidine-4-carboxylate (15.9 g), diphenylphosphoryl azide (10.4 ml) and triphenylphosphine (14.7 g) in tetrahydrofuran (400 ml) was added diisopropyl azodicarboxylate (11.0 g), and the mixture was stirred at room temperature for 30 minutes. The insoluble materials were removed by filtration. The filtrate was concentrated in vacuo, and the residue was purified by column chromatography on silica gel eluting with ethyl acetate. The eluate containing a desired product was concentrated in vacuo. The residue was dissolved in methanol (200 ml), and to the resulting solution was added a suspension of 10% palladium on carbon (4.0 g) in methanol (200 ml). The mixture was stirred under a hydrogen atmosphere at room temperature for 8 hours. After the catalyst was filtered off, the filtrate was concentrated in vacuo. The residue was dissolved in chloroform (300 ml) and extracted with 10% aqueous citric acid solution (300 ml×2). The aqueous layers were combined, adjusted to about pH 8 by addition of sodium hydrogen carbonate, and extracted with chloroform (200 ml×2). The organic layers were combined, washed with brine (100 ml×2), dried over anhydrous sodium sulfate, filtered and concentrated in vacuo to give tert-butyl 3-[1-amino-3-(butoxycarbonylamino)propyl]-4,5,6,7-tetrahydropyrazolo[1,5-a]pyrimidine-4-carboxylate (10.7 g) as a solid.

¹H-NMR (CDCl₃) δ 1.43 (9H, s), 1.53 (9H, s), 1.84-1.93 (2H, m), 2.10-2.17 (2H, m), 3.05-3.13 (1H, m), 3.31-3.41 (1H, m), 3.65-3.71 (1H, m), 3.83-3.90 (1H, m), 3.99 (1H, t, J=7.3 Hz), 4.13-4.23 (2H, m), 5.40 (1H, brs), 7.48 (1H, s)

### Preparation 25

To a solution of tert-butyl 3-[1-amino-3-(butoxycarbonylamino)propyl]-4,5,6,7-tetrahydropyrazolo[1,5-a]pyrimidine-4-carboxylate (2.0 g) in methylene chloride (10 ml) were added triethylamine (0.70 ml) and ethyl trifluoroacetate (0.60 ml), and the mixture was stirred at room temperature for 1.5 hours. To the reaction mixture was added trifluoroacetate (1.2 ml), and the mixture was stirred at the same temperature for 17 hours. To the resulting reaction mixture was added methylene chloride (20 ml), and the solution was washed successively with water (20 ml×2) and brine (20 ml). The organic layer was dried over anhydrous sodium sulfate, filtered and concentrated in vacuo to give tert-butyl 3-[3-(butoxycarbonylamino)-1-(trifluoroacetamido)propyl]-4,5,6,7-tetrahydropyrazolo[1,5-a]pyrimidine-4-carboxylate (2.3 g) as a solid.

¹H-NMR (CDCl₃) δ 1.43 (9H, s), 1.54 (9H, s), 1.85-1.95 (2H, m), 2.07-2.25 (2H, m), 2.88-2.97 (1H, m), 3.20-3.28 (1H, m), 3.34-3.43 (1H, m), 4.15-4.29 (3H, m), 5.06-5.18 (2H, m), 7.40 (1H, s), 8.67 (1H, s)

### Preparation 26

To a solution of tert-butyl 3-[3-(butoxycarbonylamino)-1-(trifluoroacetamido)propyl]-4,5,6,7-tetrahydropyrazolo[1,5-a]pyrimidine-4-carboxylate (2.2 g) in methylene chloride (6.6 ml) were added anisole (2.2 ml) and trifluoroacetic acid (6.6 ml). The mixture was stirred at room temperature for 3 hours. To the reaction mixture were added water (20 ml) and methylene chloride (20 ml), and the layers were separated. The organic layer was extracted with water (20 ml). The aqueous layer was adjusted to about pH 7 by addition of saturated aqueous sodium hydrogen carbonate solution. To the resulting solution was added a solution of di-tert-butyl dicarbonate (980 mg) in tetrahydrofuran (20 ml) and the mixture was stirred at room temperature for 17 hours. To the reaction mixture was added chloroform (50 ml), and the layers were separated. The aqueous layer was extracted with chloroform (25 ml×2). The organic layers were combined, dried over anhydrous sodium sulfate, filtered and concentrated in vacuo. The residue was triturated with hexane to give tert-butyl 3-(4,5,6,7-tetrahydropyrazolo[1,5-a]pyrimidin-3-yl)-3-(trifluoroacetamido)propylcarbamate (1.7 g) as a solid.

¹H-NMR (CDCl₃) δ 1.43 (9H, s), 2.04-2.16 (4H, m), 3.06-3.15 (1H, m), 3.15-3.27 (1H, m), 3.27-3.34 (2H, m), 4.05-4.09 (2H, m), 4.72-4.79 (1H, m), 7.18 (1H, s)

### Preparation 27

To a solution of tert-butyl 3-(4,5,6,7-tetrahydropyrazolo[1,5-a]pyrimidin-3-yl)-3-(trifluoroacetamido)propylcarbamate (1.6 g) in methanol (16 ml) was added a solution of potassium carbonate (1.2 g) in water (16 ml). The mixture was stirred at room temperature for 4 hours. Additional potassium carbonate (1.2 g) was added to the reaction mixture, and the mixture was stirred at the same temperature for 1 hour. To the reaction mixture were added hexane (20 ml) and water (20 ml). The layers were separated, and the aqueous layer was washed with diisopropyl ether (20 ml×2). The aqueous layer was extracted with chloroform (20 ml×4). The organic layers were combined, dried over anhydrous sodium sulfate, filtered and concentrated in vacuo to give tert-butyl 3-amino-3-(4,5,6,7-tetrahydropyrazolo[1,5-a]pyrimidin-3-yl)propylcarbamate (770 mg) as an oil.

¹H-NMR (CDCl₃) δ 1.44 (9H, s), 1.69-1.78 (1H, m), 1.80-1.88 (1H, m), 2.07-2.18 (2H, m), 3.10-3.26 (2H, m), 3.26-3.34 (1H, m), 3.27-3.34 (2H, m), 3.89 (1H, t, J=6.4), 4.05-4.14 (2H, m), 4.70 (1H, brs), 5.01 (1H, brs), 7.13 (1H, s)

### Preparation 28

To a solution of tert-butyl 3-amino-3-(4,5,6,7-tetrahydropyrazolo[1,5-a]pyrimidin-3-yl)propylcarbamate (770 mg) in methylene chloride (5 ml) were added triethylamine (0.37 mg) and di-tert-butyl ({[(trifluoromethyl)sulfonyl]imino}methylene)biscarbamate (1.0 g). The mixture was stirred at room temperature for 2 hours. To the reaction mixture was added ethyl acetate (40 ml). The resulting solution was washed successively with water (30 ml×2) and brine (30 ml×2). The organic layer was dried over anhydrous sodium sulfate, filtered and concentrated in vacuo. The residue was purified by column chromatography on silica gel eluting with 5% methanol/chloroform to give tert-butyl 3-[2,3-bis(tert-butoxycarbonyl)guanidino]-3-(4,5,6,7-tetrahydropyrazolo[1,5-a]pyrimidin-3-yl)propylcarbamate (900 mg) as a solid.

¹H-NMR (CDCl₃) δ 1.44 (9H, s), 1.47 (9H, s), 1.49 (18H, s), 2.02-2.18 (4H, m), 3.15-3.35 (4H, m), 4.00-4.14 (2H, m), 4.69-4.78 (2H, m), 6.47 (1H, brs), 7.20 (1H, s), 8.49 (1H, m), 11.3 (1H, brs)

### Preparation 29

To a solution of tert-butyl 3-amino-3-(4,5,6,7-tetrahydropyrazolo[1,5-a]pyrimidin-3-yl)propylcarbamate (1.98 g) in chloroform (5 ml) was added 4 M hydrogen chloride solution in dioxane (10 ml). The mixture was stirred at the room temperature for 1 hour. To the reaction mixture was added diethyl ether (20 ml). The crystalline precipitate was collected by filtration and dried in vacuo to give crude 1-(4,5,6,7-tetrahydropyrazolo[1,5-a]pyrimidin-3-yl)propane-1,3-diamine hydrochloride (1.66 g) as a solid. This product was used directly in the next step without further purification.

To a solution of the crude 1-(4,5,6,7-tetrahydropyrazolo[1,5-a]pyrimidin-3-yl)propane-1,3-diamine hydrochloride (830 mg) in chloroform (5 ml) were added triethylamine (1.40 ml) and di-tert-butyl ({[(trifluoromethyl)sulfonyl]imino}methylene)biscarbamate (1.97 g). The mixture was stirred at room temperature for 17 hours. To the reaction mixture was added ethyl acetate (50 ml), and the resulting solution was washed successively with water (40 ml×2) and brine (40 ml). The organic layer was dried over anhydrous sodium sulfate, filtered and concentrated in vacuo. The residue was purified by column chromatography on silica gel eluting with 50% hexane/ethyl acetate to give tert-butyl {3-[2,3-bis(tert-butoxycarbonyl)guanidino]-3-(4,5,6,7-tetrahydropyrazolo[1,5-a]pyrimidin-3-yl)propylamino}[(tert-butoxycarbonyl)imino]methylcarbamate (360 mg) as a solid.

¹H-NMR (CDCl₃) δ 1.47 (9H, s), 1.48 (9H, s), 1.49 (18H, s), 2.05-2.24 (4H, m), 3.17-3.37 (2H, m), 3.38-3.53 (2H, m), 3.98-4.13 (2H, m), 4.67-4.77 (1H, m), 6.60 (1H, brs), 7.23 (1H, s), 8.33 (1H, t, J=5.0 Hz), 8.53 (1H, d, J=7.3 Hz), 11.26 (1H, s), 11.45 (1H, s)

### Example 12

To a solution of 4-methoxybenzyl 70-[(Z)-2-(5-amino-1,2,4-thiadiazol-3-yl)-2-(1-tert-butoxycarbonyl-1-methylethoxyimino)acetamido]-3-chloromethyl-3-cephem-4-carboxylate (681 mg) in N,N-dimethylformamide (1.5 ml) was added N-(trimethylsilyl)acetamide (656 mg), and the mixture was stirred at room temperature for 30 minutes. To the solution was added potassium iodide (232 mg), and the mixture was stirred at room temperature for 30 minutes. To the reaction mixture was added a solution of tert-butyl 3-[2,3-bis(tert-butoxycarbonyl)guanidino]-3-(4,5,6,7-tetrahydropyrazolo[1,5-a]pyrimidin-3-yl)propylcarbamate (430 mg) in N,N-dimethylformamide (1.5 ml), and the whole mixture was stirred at 50° C. for 2 hours. To the resulting reaction mixture was added ethyl acetate (50 ml). The solution was washed successively with water (40 ml×2), 10% aqueous sodium trifluoroacetate solution (40 ml) and brine (40 ml), dried over anhydrous sodium sulfate and filtered. The filtrate was concentrated to about 5 ml in vacuo. The concentrate was poured into diisopropyl ether (80 ml), and the resulting precipitate was collected by filtration and dried in vacuo. To a solution of the solid in methylene chloride (2.8 ml) were added anisole (0.9 ml) and trifluoroacetic acid (2.8 ml). The resulting solution was stirred at room temperature for 4 hours and poured into diisopropyl ether (80 ml). The resulting precipitate was collected by filtration and dried in vacuo to give a crude product (780 mg), which was purified by preparative high-performance liquid chromatography (HPLC) utilizing an ODS column. The eluate containing a desired product was concentrated to about 20 ml in vacuo. The concentrate was further purified by preparative HPLC utilizing an ODS column eluting with 15% acetonitrile/water. The eluate was concentrated to about 10 ml in vacuo, and 0.05 mol/l sulfuric acid (1.9 ml) was added thereto. The resulting solution was lyophilized to give 7β-[(Z)-2-(5-amino-1,2,4-thiadiazol-3-yl)-2-(1-carboxy-1-methylethoxyimino)acetamido]-3-[3-(3-amino-1-guanidinopropyl)-4,5,6,7-tetrahydro-1-(pyrazolo[1,5-a]pyrimidinio)]methyl-3-cephem-4-carboxylate sulfate (83.5 mg) as an amorphous solid.

¹H-NMR (D₂O) δ 1.61 (6H, d, J=2.3 Hz), 2.05-2.19 (2H, m), 2.19-2.28 (2H, m), 3.03-3.16 (2H, m), 3.19-3.28 (1H, m), 3.32-3.48 (3H, m), 4.03-4.18 (2H, m), 4.60-4.66 (1H, m), 4.90-4.98 (1H, m), 5.18-5.26 (2H, m), 5.85 (1H, d, J=5.0), 7.92 (1H, d, J=4.1)

### Preparation 30

To a solution of 4,5,6,7-tetrahydropyrazolo[1,5-a]pyrimidine (3 g) in acetic acid (30 ml) was added di-tert-butyl (2-oxo-1,3-propanediyl)biscarbamate (8.08 g), and the mixture was stirred at 70° C. for 4.5 hours. The reaction mixture was poured into a mixture of water and ethyl acetate, and the pH was adjusted to 7 with aqueous sodium hydrogen carbonate solution. The separated organic layer was washed with brine, dried over magnesium sulfate, and evaporated in vacuo. The residue was purified by column chromatography on silica gel to give di-tert-butyl [(1Z)-2-(4,5,6,7-tetrahydropyrazolo[1,5-a]pyrimidin-3-yl)-1-propene-1,3-diyl]biscarbamate (1.39 g).

¹H-NMR (DMSO-d₆) δ 1.38 (9H, s), 1.42 (9H, s), 1.85-2.01 (2H, m), 3.08-3.18 (2H, m), 3.74 (2H, d, J=6.12 Hz), 3.98 (2H, t, J=5.96 Hz), 5.59 (1H, br-s), 6.40 (1H, d, J=10.1 Hz), 7.14 (1H, s), 7.15-7.18 (1H, m), 8.61 (1H, d, J=10.1 Hz)

### Preparation 31

To a solution of di-tert-butyl [(1Z)-2-(4,5,6,7-tetrahydropyrazolo[1,5-a]pyrimidin-3-yl)-1-propene-1,3-diyl]biscarbamate (1.38 g) in a mixture of methanol (20 ml) and tetrahydrofuran (30 ml) was added 10% palladium on carbon (700 mg), and the mixture was hydrogenated under balloon pressure for 15 hours. The reaction mixture was filtered through a bed of Celite®, and the filtrate was concentrated in vacuo to give di-tert-butyl [2-(4,5,6,7-tetrahydropyrazolo[1,5-a]pyrimidin-3-yl)-1,3-propanediyl]biscarbamate (1.03 g).

¹H-NMR (DMSO-d₆) δ 1.36 (18H, s), 1.92-2.05 (2H, m), 2.58-2.65 (1H, m), 2.95-3.05 (4H, m), 3.08-3.18 (2H, m), 3.88-3.97 (2H, m), 5.42 (1H, s), 6.48-6.65 (2H, m), 6.92 (1H, s)

### Preparation 32

To a solution of di-tert-butyl [2-(4,5,6,7-tetrahydropyrazolo[1,5-a]pyrimidin-3-yl)-1,3-propanediyl]biscarbamate (1.02 g) in methanol (5 ml) was added hydrogen chloride (4M solution in ethyl acetate, 5 ml), and the reaction mixture was stirred at room temperature for 2 hours. To the mixture was added IPE (isopropylether: 50 ml), and the resulting precipitate was collected by filtration to give 1,3-diamino-2-(4,5,6,7-tetrahydropyrazolo[1,5-a]pyrimidin-3-yl)propane dihydrochloride (692 mg).

¹H-NMR (DMSO-d₆) δ 1.98-2.09 (2H, m), 2.88-3.47 (7H, m), 3.95-4.13 (2H, m), 7.79 (1H, s)

### Preparation 33

To a mixture of 1,3-diamino-2-(4,5,6,7-tetrahydropyrazolo[1,5-a]pyrimidin-3-yl)propane dihydrochloride (700 mg) in methylene chloride (10 ml) were added di-tert-butyl ({[(trifluoromethyl)sulfonyl]imino}methylene)biscarbamate (2.25 g) and triethylamine (2.18 ml) at room temperature. The reaction mixture was stirred at room temperature for 24 hours, and the mixture was poured into a mixture of water and ethyl acetate. The separated organic layer was washed with water and brine, dried over magnesium sulfate, and evaporated in vacuo. The residue was purified by column chromatography on silica gel to give di-tert-butyl [(1E,7Z)-7-[(tert-butoxycarbonyl)amino]-11,11-dimethyl-9-oxo-4-(4,5,6,7-tetrahydropyrazolo[1,5-a]pyrimidin-3-yl)-10-oxa-2,6,8-triazadodec-7-en-1-yl-1-ylidene]biscarbamate (1.774 g).

¹H-NMR (DMSO-d₆) δ 1.40 (18H, s), 1.47 (18H, s), 1.95-2.04 (2H, m), 3.01-3.12 (1H, m), 3.22-3.33 (2H, m), 3.35-3.48 (4H, m), 3.91-3.99 (2H, m), 6.03 (1H, br-s), 7.00 (1H, s), 8.38-8.52 (2H, m), 11.51 (2H, s)

### Example 13

To a solution of 4-methoxybenzyl 7β-[(Z)-2-(5-amino-1,2,4-thiadiazol-3-yl)-2-(1-tert-butoxycarbonyl-1-methylethoxyimino)acetamido]-3-chloromethyl-3-cephem-4-carboxylate (1.3 g) in 1-methyl-2-pyrrolidone (3.9 ml) was added 1,3-bis(trimethylsilyl)urea (1.95 g), and the mixture was stirred at room temperature for 30 minutes. To the solution was added potassium iodide (443 mg), and the mixture was stirred at room temperature for 30 minutes. To the reaction mixture was added di-tert-butyl [(1E,7Z)-7-[(tert-butoxycarbonyl)amino]-11,11-dimethyl-9-oxo-4-(4,5,6,7-tetrahydropyrazolo[1,5-a]pyrimidin-3-yl)-10-oxa-2,6,8-triazadodec-7-en-1-yl-1-ylidene]biscarbamate (1.43 g), and the mixture was stirred at 35° C. for 1 hour. To the resulting reaction mixture was added ethyl acetate and the solution was washed successively with water, 10% aqueous sodium thiosulfate solution, 10% aqueous sodium trifluoroacetate solution and brine, dried over anhydrous magnesium sulfate and filtered. The filtrate was concentrated and poured into IPE, and the resulting precipitate was collected by filtration and dried in vacuo. To a solution of the solid in methylene chloride (6.48 ml) were added anisole (2.16 ml) and trifluoroacetic acid (6.48 ml), and the resulting solution was stirred at room temperature for 4 hours and poured into IPE. The resulting precipitate was collected by filtration and dried in vacuo to give a crude product, which was purified by preparative high-performance liquid chromatography utilizing an ODS column. The eluate containing a desired product was adjusted to pH 1.5 with 2M-sulfuric acid, and chromatographed on Diaion® HP-20 (Mitsubishi Chemical Corporation) eluting with 15% aqueous IPA. The eluate was concentrated to about 350 ml in vacuo, and 2M sulfuric acid (0.18 ml) was added to the solution. The solution was lyophilized to give 3-[2-{[amino(imino)methyl]amino}-1-({[amino(imino)methyl]amino}methyl)ethyl]-1-{[(6R,7R)-7-({(2Z)-2-(5-amino-1,2,4-thiadiazol-3-yl)-2-[(1-carboxy-1-methylethoxy)imino]acetyl}amino)-2-carboxy-8-oxo-5-thia-1-azabicyclo[4.2.0]oct-2-en-3-yl]methyl}-4,5,6,7-tetrahydropyrazolo[1,5-a]pyrimidin-1-ium hydrogen sulfate (424 mg).

¹H-NMR (D₂O) δ 1.61 (6H, s), 2.07-2.22 (2H, m), 3.12-3.61 (9H, m), 4.08-4.17 (2H, m), 4.85-5.21 (2H, m), 5.23 (1H, d, J=4.8 Hz), 5.84 (1H, d, J=4.8 Hz), 7.84 (1H, s)

### Preparation 34

To a solution of [1-methyl-5-(tritylamino)-1H-pyrazol-4-yl]formamide (12.47 g) in DMF (dimethylformamide: 125 ml) was added portionwise sodium hydride (60% dispersion in mineral oil, 1.43 g) under ice-cooling. The mixture was stirred at room temperature for 1 hour. To the reaction mixture were added (4-bromobutoxy)(tert-butyl)dimethylsilane (9.20 g) and NaI (5.38 g) at room temperature, and the mixture was stirred overnight at the same temperature. The resulting mixture was poured into water, and extracted with EtOAc (ethyl acetate). The organic layer was washed with brine, dried over MgSO₄, filtered and concentrated in vacuo. The residual solid was purified column chromatography on silica gel to give (4-{[tert-butyl(dimethyl)silyl]oxy}butyl)[1-methyl-5-(tritylamino)-1H-pyrazol-4-yl]formamide (7.80 g) as an amorphous solid.

¹H-NMR (DMSO-d₆) δ 0.00 (6H, s), 0.83 (9H, s), 1.20-1.40 (4H, m), 2.70-2.95 (2H, m), 2.74 (3H, s), 3.45-3.55 (2H, m), 6.02 (1H, s), 7.00-7.35 (16H, m), 7.59 (1H, s)

### Preparation 35

To a solution of (4-{[tert-butyl(dimethyl)silyl]oxy}butyl)[1-methyl-5-(tritylamino)-1H-pyrazol-4-yl]formamide (7.00 g) in THF (70 ml) was added a solution of 1.0 M tetrabutylammonium fluoride in THF (13 ml). The mixture was stirred at room temperature for 2 hours. To the resulting mixture was added water, and the mixture was extracted with EtOAc. The organic layer was washed with brine, dried over MgSO₄, filtered and concentrated in vacuo. The residual solid was purified by column chromatography to give (4-hydroxybutyl)[1-methyl-5-(tritylamino)-1H-pyrazol-4-yl]formamide (4.68 g) as an amorphous solid.

¹H-NMR (DMSO-d₆) δ 1.13-1.38 (4H, m), 2.70-2.90 (2H, m), 2.76 (3H, s), 3.25-3.40 (2H, m), 4.35 (1H, t, J=5.2 Hz), 6.03 (1H, s), 7.00-7.35 (16H, m, J=7.58 Hz), 7.58 (1H, s)

### Preparation 36

To a solution of (4-hydroxybutyl)[1-methyl-5-(tritylamino)-1H-pyrazol-4-yl]formamide (455 mg) in CH₂Cl₂ (9 ml) were added triethylamine (0.28 ml) and methanesulfonyl chloride (0.103 ml) under ice-cooling. The mixture was stirred at room temperature for 0.5 hour. To the resulting mixture was added water, and the mixture was extracted with EtOAc. The organic layer was washed with brine, dried over MgSO₄, filtered and concentrated in vacuo. The residual solid was purified by column chromatography to give 4-{formyl[1-methyl-5-(tritylamino)-1H-pyrazol-4-yl]amino/butyl methanesulfonate (484 mg) as an amorphous solid.

¹H-NMR (DMSO-d₆) δ 1.17-1.38 (2H, m), 1.38-1.60 (2H, m), 2.76 (3H, s), 2.89 (2H, t, J=7.1 Hz), 3.14 (3H, s), 4.07-4.18 (2H, m, J=6.04 Hz), 6.04 (1H, s), 7.02-7.32 (16H, m), 7.60 (1H, s)

### Preparation 37

To a solution of 4-{formyl[1-methyl-5-(tritylamino)-1H-pyrazol-4-yl]amino}butyl methanesulfonate (5.00 g) in DMF (50 ml) was added sodium azide (1.22 g). The mixture was stirred at 80° C. for 2 hours. After cooling to room temperature, water was added to the resulting mixture, and the mixture was extracted with EtOAc. The organic layer was washed with water and brine, dried over MgSO₄, filtered and concentrated in vacuo to give (4-azidobutyl)[1-methyl-5-(tritylamino)-1H-pyrazol-4-yl]formamide (4.50 g) as an amorphous solid.

¹H-NMR (DMSO-d₆) δ 1.08-1.45 (4H, m), 2.76 (3H, s), 2.80-2.95 (2H, m), 3.20-3.30 (2H, m), 6.04 (1H, s), 7.02-7.32 (16H, m), 7.60 (1H, s)

### Preparation 38

To a solution of (4-azidobutyl)[1-methyl-5-(tritylamino)-1H-pyrazol-4-yl]formamide (418 mg) in THF (4 ml) was added 10% palladium on carbon (200 mg). The reaction mixture was stirred under H₂ atmosphere at 40° C. for 2 hours. The catalyst was filtered off, and the catalyst was washed with THF. The combined filtrate was concentrated in vacuo. The residue was diluted with CH₂Cl₂ (4 ml), and to this solution were added triethylamine (0.15 ml) and trityl chloride (292 mg). After stirring at room temperature for 2 hours, water was added to the resulting mixture and the mixture was extracted with EtOAc. The organic layer was washed with water and brine, dried over MgSO₄, filtered and concentrated in vacuo. The residue was purified with column chromatography on silica gel to give [1-methyl-5-(tritylamino)-1H-pyrazol-4-yl][4-(tritylamino)butyl]formamide (192 mg) as an amorphous solid.

¹H-NMR (DMSO-d₆) δ 0.95-1.45 (4H, m), 1.72-1.95 (2H, m), 2.60-2.90 (2H, m), 2.74 (3H, s), 6.02 (1H, s), 7.00-7.48 (31H, m), 7.63 (1H, s)

### Preparation 39

To the solution of [1-methyl-5-(tritylamino)-1H-pyrazol-4-yl][4-(tritylamino)butyl]formamide (3.4 g) in MeOH (34 ml) was added concentrated HCl (4.2 ml). The reaction mixture was stirred at room temperature for 8 hours. To the reaction mixture were added NaHCO₃ (2.23 g) and water. The volatiles were evaporated, and to the residue was added NaHCO₃ (223 mg) and the mixture was extracted with EtOAc. The organic phase was dried over MgSO₄, filtered and concentrated in vacuo to give crude 1-methyl-N-4-[4-(tritylamino)butyl]-1H-pyrazole-4,5-diamine. This material was used for the next step without further purification.

### Preparation 40

To a solution of crude 1-methyl-N-4-[4-(tritylamino)butyl]-1H-pyrazole-4,5-diamine (2.15 g) in THF (43 ml) were added di-tert-butyl ({[(trifluoromethyl)sulfonyl]imino}methylene)biscarbamate (2.38 g) and triethylamine (1.27 ml). The reaction mixture was stirred at 50° C. overnight. To the resulting solution was added water, and the mixture was extracted with EtOAc. The organic layer was washed with brine, dried over MgSO₄, filtered and concentrated in vacuo. The residue was purified by column chromatography on silica gel to give di-tert-butyl ((E)-{(5-amino-1-methyl-1H-pyrazol-4-yl)[4-(tritylamino)butyl]amino}methylylidene)biscarbamate (510 mg) as an amorphous solid.

¹H-NMR (DMSO-d₆) δ 1.20-1.55 (4H, m), 1.33 (9H, s), 1.35 (9H, s), 1.80-2.00 (2H, m), 3.30-3.48 (2H, m), 3.50 (3H, s), 5.07 (2H, s), 5.76 (1H, s), 7.02 (1H, s), 7.11-7.45 (16H, m), 8.57 (1H, br-s), 9.15 (1H, s), 11.52 (1H, s)

### Example 14

5-Amino-4-{(4-aminobutyl)[amino(imino)methyl]amino}-2-{[(6R,7R)-7-({(2Z)-2-(5-amino-1,2,4-thiadiazol-3-yl)-2-[(1-carboxy-1-methylethoxy)imino]acetyl}amino)-2-carboxy-8-oxo-5-thia-1-azabicyclo[4.2.0]oct-2-en-3-yl]methyl}-1-methyl-1H-pyrazol-2-ium hydrogen sulfate was synthesized according to a similar manner to that of Example 13.

¹H-NMR (D₂O) δ 1.55-1.78 (4H, m), 1.61 (6H, s), 2.90-3.10 (2H, m), 3.31 (1H, d, J=17.8 Hz), 3.40-3.88 (5H, m), 3.75 (3H, s), 4.75-5.35 (3H, m), 5.75-5.95 (1H, m), 8.05-8.25 (1H, m)

### Preparation 41

To a suspension of 4,5,6,7-tetrahydropyrazolo[1,5-a]pyrimidin-3-amine sulfate (10 g) in tetrahydrofuran (100 ml) were added N,N-diisopropylethylamine (16.22 ml) and benzyl {2-[(2,5-dioxo-1-pyrrolidinyl)oxy]-2-oxoethyl}carbamate (14.26 g) under ice-cooling. The reaction mixture was stirred at room temperature for 24 hours and poured into a mixture of water and ethyl acetate. To the mixture were added sodium hydrogen carbonate and sodium chloride. The organic layer was separated, dried over magnesium sulfate and filtered. The filtrate was evaporated in vacuo to give benzyl [2-oxo-2-(4,5,6,7-tetrahydropyrazolo[1,5-a]pyrimidin-3-ylamino)ethyl]carbamate (13.51 g).

¹H-NMR (DMSO-d₆) δ 1.88-2.02 (2H, m), 3.12-3.21 (2H, m), 3.72 (2H, d, J=6.1 Hz), 3.94 (2H, t, J=6.02 Hz), 5.04 (2H, s), 5.43 (1H, s), 7.05 (1H, s), 7.32-7.41 (5H, m), 7.50 (1H, t, J=6.1 Hz), 9.23 (1H, s)

### Preparation 42

To a solution of benzyl [2-oxo-2-(4,5,6,7-tetrahydropyrazolo[1,5-a]pyrimidin-3-ylamino)ethyl]carbamate (7 g) in methanol (140 ml) was added 10% palladium on carbon (700 mg), and the mixture was hydrogenated under balloon pressure for 4 hours. The reaction mixture was filtered through a bed of Celite®, and the filtrate was concentrated in vacuo to give 2-amino-N-(4,5,6,7-tetrahydropyrazolo[1,5-a]pyrimidin-3-yl)acetamide (4 g).

¹H-NMR δ 1.90-2.04 (2H, m), 3.15-3.42 (4H, m), 3.94 (2H, t, J=6.04 Hz), 5.55 (1H, s), 7.15 (1H, s)

### Preparation 43

To a suspension of 2-amino-N-(4,5,6,7-tetrahydropyrazolo[1,5-a]pyrimidin-3-yl)acetamide (4 g) in methylene chloride (160 ml) was added diisobutylaluminum hydride (1.01 M solution in toluene) (122 ml) at room temperature, and under a nitrogen atmosphere the reaction mixture was refluxed for 5 hours. After cooling to 0° C. to 5° C., the mixture was diluted with additional methylene chloride (160 ml), and to the mixture was added sodium fluoride (24 g) in one portion. To the mixture was added dropwise water (4 ml) under ice-cooling, and then warmed to room temperature. After stirring at room temperature for 30 minutes, di-tert-butyl ({[(trifluoromethyl)sulfonyl]imino}methylene)biscarbamate (17.64 g) was added to the mixture. The mixture was refluxed for 18 hours, and the insoluble materials were filtered off. The filtrate was washed with water and brine, dried over magnesium sulfate and evaporated in vacuo. The residue was purified by chromatography on silica gel to give di-tert-butyl [(1E,6Z)-6-[(tert-butoxycarbonyl)amino]-10,10-dimethyl-8-oxo-2-(4,5,6,7-tetrahydropyrazolo[1,5-a]pyrimidin-3-yl)-9-oxa-2,5,7-triazaundec-6-en-1-yl-1-ylidene]biscarbamate (650 mg).

¹H-NMR (DMSO-d₆) δ 1.32-1.51 (36H, m), 1.92-2.01 (2H, m), 3.11-3.20 (2H, m), 3.31-3.55 (2H, m), 3.60-3.71 (2H, m), 3.89-3.98 (2H, m), 5.78 (1H, br-s), 7.09 (1H, s), 8.32 (1H, m), 11.43 (1H, s)

### Example 15

3-[[Amino(imino)methyl](2-{[amino(imino)methyl]amino}ethyl)amino]-1-{[(6R,7R)-7-({(2Z)-2-(5-amino-1,2,4-thiadiazol-3-yl)-2-[(1-carboxy-1-methylethoxy)imino]acetyl}amino)-2-carboxy-8-oxo-5-thia-1-azabicyclo[4.2.0]oct-2-en-3-yl]methyl}-4,5,6,7-tetrahydropyrazolo[1,5-a]pyrimidin-1-ium hydrogen sulfate was synthesized according to a similar manner to that of Example 13.

¹H-NMR (D₂O) δ 1.58 (6H, s), 2.12-2.25 (2H, m), 3.13-3.92 (8H, m), 4.09-4.21 (2H, m), 4.65-4.98 (2H, m), 5.27 (1H, m), 5.83 (1H, d, J=4.74 Hz), 7.98-8.18 (1H, m)

### Example 1-6

To a solution of 4-methoxybenzyl 70-[(Z)-2-(5-amino-1,2,4-thiadiazol-3-yl)-2-(1-tert-butoxycarbonyl-1-methylethoxyimino)acetamido]-3-chloromethyl-3-cephem-4-carboxylate (477 mg) in N,N-dimethylformamide (1.0 ml) was added N-(trimethylsilyl)acetamide (459 mg), and the mixture was stirred at room temperature for 30 minutes. To the solution was added potassium iodide (162 mg), and the mixture was stirred at room temperature for 30 minutes. To the reaction mixture was added a solution of tert-butyl {3-[2,3-bis(tert-butoxycarbonyl)guanidino]-3-(4,5,6,7-tetrahydropyrazolo[1,5-a]pyrimidin-3-yl)propylamino}[(tert-butoxycarbonyl)imino]methylcarbamate (340 mg) in N,N-dimethylformamide (1.0 ml), and the whole mixture was stirred at 50° C. for 3 hours. To the resulting reaction mixture was added ethyl acetate (30 ml), and the solution was washed successively with water (25 ml×2), 10% aqueous sodium trifluoroacetate solution (25 ml) and brine (25 ml), dried over anhydrous sodium sulfate and filtered. The filtrate was concentrated to about 3 ml in vacuo. The concentrate was poured into diisopropyl ether (40 ml) and the resulting precipitate was collected by filtration and dried in vacuo. To a solution of the solid in methylene chloride (2.0 ml) were added anisole (0.66 ml) and trifluoroacetic acid (2.0 ml). The resulting solution was stirred at room temperature for 4 hours and poured into diisopropyl ether (40 ml). The resulting precipitate was collected by filtration and dried in vacuo to give a crude product (580 mg), which was purified by preparative high-performance liquid chromatography (HPLC) utilizing an ODS column. The first eluate containing a desired product was concentrated to about 20 ml in vacuo. The concentrate was further purified by preparative HPLC utilizing an ODS column eluting with 20% acetonitrile/water. The eluate was concentrated to about 10 ml in vacuo, and 0.05 M sulfuric acid (0.7 ml) was added thereto. The resulting solution was lyophilized to give 70-[(Z)-2-(5-amino-1,2,4-thiadiazol-3-yl)-2-(1-carboxy-1-methylethoxyimino)acetamido]-3-[3-(1,3-diguanidinopropyl)-4,5,6,7-tetrahydro-1-(pyrazolo[1,5-a]pyrimidinio)]methyl-3-cephem-4-carboxylate sulfate (29 mg) as an amorphous solid.

The second eluate was worked up as described above (0.05 mol/l sulfuric acid: 0.3 ml) to give 7β-[(Z)-2-(5-amino-1,2,4-thiadiazol-3-yl)-2-(1-carboxy-1-methylethoxyimino)acetamido]-3-[3-(1,3-diguanidinopropyl)-4,5,6,7-tetrahydro-1-(pyrazolo[1,5-a]pyrimidinio)]methyl-3-cephem-4-carboxylate sulfate (19 mg) as an amorphous solid.

¹H-NMR (D₂O) δ 1.62 (3H, s), 1.62 (3H, s), 2.05-2.23 (4H, m) 3.20-3.49 (6H, m), 4.04-4.22 (2H, m), 4.64 (1H, t, J=7.3 Hz), 4.95 (1H, d, J=16.5 Hz), 5.21 (1H, d, J=16.5 Hz), 5.26 (1H, d, J=4.6 Hz), 5.85 (1H, d, J=4.6 Hz), 7.91 (1H, s)

¹H-NMR (D₂O) δ 1.63 (3H, s), 1.63 (3H, s), 2.05-2.23 (4H, m), 3.20-3.49 (6H, m), 4.08-4.19 (2H, m), 4.64 (1H, t, J=7.3 Hz), 4.90 (1H, d, J=15.6 Hz), 5.19 (1H, d, J=15.6 Hz), 5.26 (1H, d, J=5.0 Hz), 5.85 (1H, d, J=5.0 Hz), 7.90 (1H, s)

### Example 17

To a solution of 70-[(Z)-2-(5-amino-1,2,4-thiadiazol-3-yl)-2-(1-carboxy-1-methylethoxyimino)acetamido]-3-{3-[1-(2-aminoethyl)guanidino]-4,5,6,7-tetrahydro-1-(pyrazolo[1,5-a]pyrimidinio)}methyl-3-cephem-4-carboxylate sulfate (395 mg) in water (8 ml) were added potassium carbonate (380 mg) and ethyl acetimidate hydrochloride (880 mg). The mixture was stirred at room temperature for 18 hours. The reaction mixture was purified by preparative high-performance liquid chromatography (HPLC) utilizing an ODS column. The eluate containing a desired product was concentrated to about 20 ml in vacuo. The concentrate was further purified by preparative HPLC utilizing an ODS column eluting with 20% acetonitrile/water. The eluate was concentrated to about 10 ml in vacuo, and 0.05 M sulfuric acid (4.6 ml) was added thereto. The resulting solution was lyophilized to give 70-[(Z)-2-(5-amino-1,2,4-thiadiazol-3-yl)-2-(1-carboxy-1-methylethoxyimino)acetamido]-3-[3-{1-[2-(acetimidoylamino)ethyl]guanidino}-4,5,6,7-tetrahydro-1-(pyrazolo[1,5-a]pyrimidinio)]methyl-3-cephem-4-carboxylate sulfate (186 mg) as an amorphous solid.

¹H-NMR (D₂O) δ 1.63 (6H, s), 2.16-2.25 (2H, m), 2.23 (3H, s), 3.31-4.01 (8H, m), 4.11-4.27 (2H, m), 4.92-5.01 (1H, m), 5.10-5.25 (1H, m), 5.29 (1H, d, J=4.6 Hz), 5.85 (1H, d, J=4.6 Hz), 8.03-8.25 (1H, m)

### Example 18

A solution of 7β-[(Z)-2-(5-amino-1,2,4-thiadiazol-3-yl)-2-(1-carboxy-1-methylethoxyimino)acetamido]-3-{3-[1-(2-aminoethyl)guanidino]-4,5,6,7-tetrahydro-1-(pyrazolo[1,5-a]pyrimidinio)}methyl-3-cephem-4-carboxylate sulfate (395 mg) in phosphate buffer solution (pH 6.8, 100 ml) was stirred at 37° C. for 24 hours. The reaction mixture was purified by preparative high-performance liquid chromatography (HPLC) utilizing an ODS column. The eluate containing a desired product was concentrated to about 20 ml in vacuo. The concentrate was further purified by preparative HPLC utilizing an ODS column eluting with 20% acetonitrile/water. The eluate was concentrated to about 10 ml in vacuo, and 0.05 M sulfuric acid (2.0 ml) was added thereto. The resulting solution was lyophilized to give 7β-[(Z)-2-(5-amino-1,2,4-thiadiazol-3-yl)-2-(1-carboxy-1-methylethoxyimino)acetamido]-3-[3-(2-iminoimidazolidin-1-yl)-4,5,6,7-tetrahydro-1-(pyrazolo[1,5-a]pyrimidinio)]methyl-3-cephem-4-carboxylate sulfate (73 mg) as an amorphous solid.

¹H-NMR (D₂O) δ 1.63 (3H, s), 1.63 (3H, s), 2.12-2.25 (2H, m), 3.27 (1H, d, J=18.3 Hz), 3.36-3.46 (2H, m), 3.59 (1H, d, J=18.3 Hz), 3.80 (2H, t, J=8.7 Hz), 3.88-3.99 (2H, m), 4.17 (1H, t, J=6.0 Hz), 4.98 (1H, d, J=15.6 Hz), 5.22 (1H, d, J=15.6 Hz), 5.28 (1H, d, J=5.0 Hz), 5.88 (1H, d, J=5.0 Hz), 8.12 (1H, s)

### Example 19

To a solution of 7β-[(Z)-2-(5-amino-1,2,4-thiadiazol-3-yl)-2-(1-carboxy-1-methylethoxyimino)acetamido]-3-{3-[1-(2-aminoethyl)guanidino]-4,5,6,7-tetrahydro-1-(pyrazolo[1,5-a]pyrimidinio)}methyl-3-cephem-4-carboxylate sulfate (395 mg) in water (8 ml) were added potassium carbonate (380 mg) and isopropyl formimidate hydrochloride (880 mg). The mixture was stirred at room temperature for 18 hours. The reaction mixture was purified by preparative high-performance liquid chromatography (HPLC) utilizing an ODS column. The eluate containing a desired product was concentrated to about 20 ml in vacuo. The concentrate was further purified by preparative HPLC utilizing an ODS column eluting with 20% acetonitrile/water. The eluate was concentrated to about 10 ml in vacuo, and 0.05 M sulfuric acid (1.2 ml) was added thereto. The resulting solution was lyophilized to give 7β-[(Z)-2-(5-amino-1,2,4-thiadiazol-3-yl)-2-(1-carboxy-1-methylethoxyimino)acetamido]-3-[3-{1-[2-(formimidoylamino)ethyl]guanidino}-4,5,6,7-tetrahydro-1-(pyrazolo[1,5-a]pyrimidinio)}methyl-3-cephem-4-carboxylate sulfate (54 mg) as an amorphous solid.

¹H-NMR (D₂O) δ 1.63 (6H, s), 2.12-2.26 (2H, m), 3.27-4.05 (8H, m), 4.10-4.27 (2H, m), 4.89-5.00 (1H, m), 5.09-5.26 (1H, m), 5.29 (1H, d, J=4.1 Hz), 5.85 (1H, d, J=4.1 Hz), 8.04-8.23 (1H, m)

### Preparation 44

To a stirred solution of N-[1-(2-hydroxyethyl)-5-(tritylamino)-1H-pyrazol-4-yl]formamide (4.3 g) in DMF (43 mL) was added sodium hydride (459 mg, 60% oil suspension) under nitrogen at 0° C., and the mixture was stirred for 15 minutes at the same temperature. A solution of 3-bromo-N-tritylpropan-1-amine (4.36 g) in DMF (10 mL) and NaI (1.72 g) were added to the mixture, and the mixture was stirred for 2 hours with warming to room temperature. 10% Aqueous KHSO₄ solution was added to the reaction solution, and the mixture was extracted with EtOAc. The organic layer was washed with water and brine, and dried over MgSO₄. The organic solvent was removed under reduced pressure to give a residual oil, which was purified with open column chromatography (SiO₂; CH₂Cl₂→CH₂Cl₂: EtOAc=2:1) to give N-[1-(2-hydroxyethyl)-5-(tritylamino)-1H-pyrazol-4-yl]-N-[3-(tritylamino)propyl]formamide (4.52 g).

¹H-NMR (CDCl₃) δ 2.7-3.5 (10H, m), 4.7-4.9 (1H, m), 5.48 & 6.07 (1H, s), 7.0-7.6 (33H, m)

ESI Mass: 710.3 [M−H]− (negative)

### Preparation 45

To a solution of [N-[1-(2-hydroxyethyl)-5-(tritylamino)-1H-pyrazol-4-yl]-N-[3-(tritylamino)propyl]formamide (4.4 g) in MeOH (45 ml) was added concentrated hydrochloric acid (5.3 ml). The reaction mixture was stirred at room temperature overnight. To the reaction mixture was added NaHCO₃ (5.3 g) and the volatiles were removed by evaporation. The residue was extracted with EtOAc. The organic phase was dried over MgSO₄, filtered and concentrated in vacuo to give crude 2-(5-amino-4-{[3-(tritylamino)propyl]amino}-1H-pyrazol-1-yl)ethanol. This material was used for the next step without further purification.

### Preparation 46

To a solution of crude 2-(5-amino-4-{[3-(tritylamino)propyl]amino}-1H-pyrazol-1-yl)ethanol (1.4 g) in THF (30 ml) was added di-tert-butyl ({[(trifluoromethyl)sulfonyl]imino}methylene)biscarbamate (1.86 g). The reaction mixture was stirred for 4 hours at room temperature. Additional di-tert-butyl ({[(trifluoromethyl)sulfonyl]imino}methylene)biscarbamate (620 mg) was added to the mixture, and the mixture was stirred overnight at the same temperature. To the resulting solution was added water, and the solution was extracted with EtOAc. The organic layer was washed with brine, dried over MgSO₄, filtered and concentrated in vacuo. The residue was purified by column chromatography on silica gel eluting with EtOAc to give di-tert-butyl [(E)-{[5-amino-1-(2-hydroxyethyl)-1H-pyrazol-4-yl][3-(tritylamino)propyl]amino}methylylidene]biscarbamate (1.39 g) as an amorphous solid.

¹H-NMR (DMSO-d₆) δ 1.30 (9H, s), 1.32 (9H, s), 1.5-1.7 (2H, m), 1.8-2.0 (2H, m), 3.4-3.7 (4H, m), 3.8-4.0 (2H, m), 4.8-5.0 (3H, m), 6.94 (1H, s), 7.1-7.5 (16H, m), 8.5-8.7 (1H, m)

ESI Mass: 706.3 [M+H]+ (positive)

### Example 20

To a solution of 4-methoxybenzyl (6R,7R)-7-({(2Z)-2-(5-amino-1,2,4-thiadiazol-3-yl)-2-[(2-tert-butoxy-1,1-dimethyl-2-oxyethoxy)imino]acetyl}amino)-3-(chloromethyl)-8-oxo-5-thia-1-azabicyclo[4.2.0]oct-2-ene-2-carboxylate (1.3 g) in DMF (3.9 ml) was added 1,3-bis(trimethylsilyl)urea (1.95 g), and the mixture was stirred at room temperature for 40 minutes. To the solution was added potassium iodide (443 mg) and the mixture was stirred at room temperature for 40 minutes. To the reaction mixture was added di-tert-butyl [(E)-{[5-amino-1-(2-hydroxyethyl)-1H-pyrazol-4-yl][3-(tritylamino)propyl]amino}methylylidene]biscarbamate (1.37 g), and the mixture was stirred at 38° C. overnight. To the resulting reaction mixture was added EtOAc, and the solution was washed successively with water, 10% aqueous sodium thiosulfate solution, 10% aqueous sodium trifluoroacetate solution and brine, dried over anhydrous magnesium sulfate and filtered. The filtrate was concentrated and the residue was poured into IPE, and the resulting precipitate was collected by filtration and dried in vacuo. The obtained solid was dissolved in methylene chloride (6.48 ml), and anisole (2.16 ml) and trifluoroacetic acid (6.48 ml) were added to the solution. The resulting solution was stirred at room temperature for 3 hours and poured into IPE. The resulting precipitate was collected by filtration and dried in vacuo to give a crude product, which was purified by preparative high-performance liquid chromatography utilizing ODS column. The pH of the eluate containing the desired product was adjusted to 1.5 with 2M-sulfuric acid, and the eluate was chromatographed on Diaion® HP-20 (Mitsubishi Chemical Corporation) eluting with 15% aqueous IPA. The eluate was concentrated to about 350 ml in vacuo. To the concentrated solution was added an equivalent amount of 2M-sulfuric acid, and the mixture was lyophilized to give 5-amino-4-{[amino(imino)methyl](3-aminopropyl)amino}-2-{[(6R,7R)-7-({(2Z)-2-(5-amino-1,2,4-thiadiazol-3-yl)-2-[(1-carboxy-1-methylethoxy)imino]acetyl}amino)-2-carboxy-8-oxo-5-thia-1-azabicyclo[4.2.0]oct-2-en-3-yl]methyl}-1-(2-hydroxyethyl)-1H-pyrazol-2-ium hydrogen sulfate.

¹H-NMR (D₂O) δ 1.61 (6H, s), 1.8-2.2 (2H, m), 2.9-3.1 (2H, m), 3.2-3.8 (4H, m), 3.8-4.0 (2H, m), 4.3-4.5 (2H, m), 4.8-5.2 (2H, m), 5.2-5.3 (1H, m), 5.8-5.9 (1H, m), 8.1-8.3 (1H, m)

### Preparation 47

To a stirred solution of N-[1-methyl-5-(tritylamino)-1H-pyrazol-4-yl]formamide (8 g) in DMF (60 mL) was added NaH (920 mg, 60% oil suspension) under nitrogen at 0° C., and the mixture was stirred for 15 minutes at the same temperature. A solution of (2-bromoethyl)tritylamine (8.43 g) in DMF (20 mL) and NaI (3.45 g) were added to the mixture, and the mixture was stirred for 2 hours with warming to room temperature. 10% Aqueous KHSO₄ was added to the reaction solution and the mixture was extracted with EtOAc. The organic layer was washed with water and brine and dried over MgSO₄. The organic solvent was removed under reduced pressure to give a residual oil, which was purified with open column chromatography (SiO₂; CH₂Cl₂ CH₂Cl₂: EtOAc=2:1) to give N-[1-methyl-5-(tritylamino)-1H-pyrazol-4-yl]-N-[2-(tritylamino)ethyl]formamide (11.5 g).

ESI Mass: 691.1 [M+Na]+ (positive)

¹H-NMR (CDCl₃) δ 2.5-3.3 (4H, m), 2.57 & 2.71 (3H, s), 5.24 & 5.96 (1H, s), 6.8-7.5 (32H, m), 7.64 & 7.70 (1H, s)

### Preparation 48

To a solution of N-[1-methyl-5-(tritylamino)-1H-pyrazol-4-yl]-N-[2-(tritylamino)ethyl]formamide (3 g) in MeOH (30 ml) was added concentrated hydrochloric acid (3.7 ml). The reaction mixture was stirred at room temperature overnight. To the reaction mixture was added NaHCO₃ (3.77 g) and the volatiles were removed by evaporation. The residue was extracted with EtOAc. The organic phase was dried over MgSO₄, filtered and concentrated in vacuo to give crude 1-methyl-N-4-[2-(tritylamino)ethyl]-1H-pyrazole-4,5-diamine. This material was used for the next step without further purification.

### Preparation 49

To a solution of crude 1-methyl-N-4-[2-(tritylamino)ethyl]-1H-pyrazole-4,5-diamine (1.79 g) in THF (36 ml) were added di-tert-butyl ({[(trifluoromethyl)sulfonyl]imino}methylene)biscarbamate (3.51 g) and triethylamine (1.88 ml). The reaction mixture was stirred at room temperature overnight. To the resulting solution was added water, and the mixture was extracted with EtOAc. The organic layer was washed with brine, dried over MgSO₄, filtered and concentrated in vacuo. The residue was purified by column chromatography on silica gel to give di-tert-butyl [(E)-{(5-amino-1-methyl-1H-pyrazol-4-yl)[2-(tritylamino)ethyl]amino}methylylidene]biscarbamate (450 mg) as an amorphous solid.

ESI Mass: 638.3 [M−H]− (negative)

¹H-NMR (DMSO-d₆) δ 1.35 (18H, s), 2.1-2.2 (2H, m), 3.46 (3H, s), 3.5-3.7 (2H, m), 5.14 (2H, s), 6.74 (1H, s), 7.1-7.5 (17H, m)

### Example 21

To a solution of 4-methoxybenzyl (6R,7R)-7-({(2Z)-2-(5-amino-1,2,4-thiadiazol-3-yl)-2-[(2-tert-butoxy-1,1-dimethyl-2-oxyethoxy)imino]acetyl}amino)-3-(chloromethyl)-8-oxo-5-thia-1-azabicyclo[4.2.0]oct-2-ene-2-carboxylate (1 g) in DMF (3 ml) was added 1,3-bis(trimethylsilyl)urea (1.5 g), and the mixture was stirred at room temperature for 30 minutes. To the solution was added potassium iodide (341 mg), and the mixture was stirred at room temperature for 30 minutes. To the reaction mixture was added di-tert-butyl [(E)-{(5-amino-1-methyl-1H-pyrazol-4-yl)[2-(tritylamino)ethyl]amino}methylylidene]biscarbamate (986 mg), and the mixture was stirred at 38° C. overnight. To the resulting reaction mixture was added ethyl acetate, and the solution was washed successively with water, 10% aqueous sodium thiosulfate solution, 10% aqueous sodium trifluoroacetate solution and brine, dried over anhydrous magnesium sulfate and filtered. The filtrate was concentrated and poured into IPE, and the resulting precipitate was collected by filtration and dried in vacuo. To a solution of the solid in methylene chloride (6 ml) were added anisole (2 ml) and trifluoroacetic acid (6 ml), and the resulting solution was stirred at room temperature for 3 hours and poured into IPE. The resulting precipitate was collected by filtration and dried in vacuo to give a crude product, which was purified by preparative high-performance liquid chromatography utilizing an ODS column. The pH of the eluate containing a desired product was adjusted to 1.5 with 2M-sulfuric acid, and the eluate was chromatographed on Diaion® HP-20 (Mitsubishi Chemical Corporation) eluting with 15% aqueous IPA. The purified eluate was concentrated to about 350 ml in vacuo. To the solution was added an equivalent amount of 2M-sulfuric acid, and the solution was lyophilized to give 5-amino-4-{(2-aminoethyl)[amino(imino)methyl]amino}-2-{[(6R,7R)-7-({(2Z)-2-(5-amino-1,2,4-thiadiazol-3-yl)-2-[(1-carboxy-1-methylethoxy)imino]acetyl}amino)-2-carboxy-8-oxo-5-thia-1-azabicyclo[4.2.0]oct-2-en-3-yl]methyl}-1-methyl-1H-pyrazol-2-ium hydrogen sulfate.

¹H-NMR (D₂O) δ 1.61 (6H, s), 3.2-3.4 (3H, m), 3.5-4.2 (3H, m), 4.7-5.2 (2H, m), 5.28 (1H, d, J=4.8 Hz), 5.86 (1H, d, J=4.6 Hz), 8.2-8.3 (1H, m)

### INDUSTRIAL APPLICABILITY

The present invention provides a novel cephem compound and a pharmaceutically acceptable salt thereof, and a process for preparing the same. The cephem compound and the pharmaceutically acceptable salt thereof show excellent antimicrobial activity and are useful for medicaments such as antibacterial agents.

This application is based on the U.S. Provisional Application Ser. No. 60/782,536 filed on Mar. 16, 2006, and the contents of which are incorporated hereinto by reference.

1. A compound of the formula [I]: 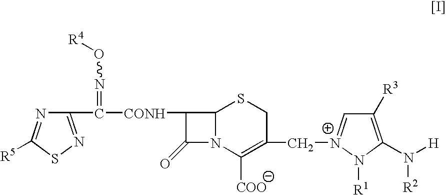
wherein
R¹ is lower alkyl or hydroxy(lower)alkyl, and
R² is hydrogen or amino protecting group, or
R¹ and R² are bonded together to form lower alkylene;
R³ is a group represented by 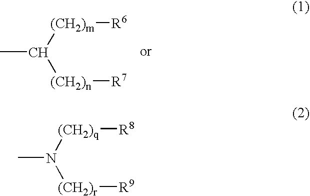
wherein
R⁶ and R⁷ are independently optionally protected amino or optionally protected guanidino, provided that R⁶ and R⁷ are not simultaneously amino groups,
m and n are independently an integer of 0 to 6,
R⁸ and R⁹ are independently optionally protected amino, optionally protected imino(lower)alkylamino, optionally protected guanidino or optionally protected amidino, and
q and r are independently an integer of 0 to 6, or
R⁸ and R⁹, together with the adjacent alkylene(s) and the nitrogen atom, form saturated nitrogen-containing heterocycle optionally having substituent(s),
R⁴ is lower alkyl optionally substituted with optionally protected carboxy; and
R⁵ is amino or protected amino,
or a pharmaceutically acceptable salt thereof.

2. The compound of claim 1, wherein
R¹ is lower alkyl or hydroxy(lower)alkyl, and
R² is hydrogen, aryl(lower)alkyl or acyl, or
R¹ and R² are bonded together to form lower alkylene;
R⁴ is lower alkyl optionally substituted with carboxy or esterified carboxy;
R⁵ is amino, aryl(lower)alkylamino, lower alkanoylamino or lower alkoxycarbonylamino;
R⁶ and R⁷ are independently guanidino or amino; and
R⁸ and R⁹ are independently amidino, amino, imino(lower)alkylamino or guanidino, or
R⁸ and R⁹, together with the adjacent alkylene(s) and the nitrogen atom, form saturated nitrogen-containing heterocycle optionally having substituent(s),
or a pharmaceutically acceptable salt thereof.

3. The compound of claim 2, wherein
R¹ is lower alkyl or hydroxy(lower)alkyl, and
R² is hydrogen, aryl(lower)alkyl, lower alkanoyl or lower alkoxycarbonyl, or
R¹ and R² are bonded together to form lower alkylene;
R⁴ is lower alkyl optionally substituted with carboxy or esterified carboxy;
R⁵ is amino, aryl(lower)alkylamino or acylamino;
R⁶ and R⁷ are independently guanidino or amino; and
R⁸ and R⁹ are independently amidino, amino, imino(lower)alkylamino or guanidino, or
R⁸ and R⁹, together with the adjacent alkylene(s) and the nitrogen atom, form 5- or 6-membered saturated nitrogen-containing heterocycle optionally having substituent(s),
or a pharmaceutically acceptable salt thereof.

4. The compound of claim 3, wherein
R¹ is lower alkyl,
R² is hydrogen, or
R¹ and R² are bonded together to form lower alkylene;
R⁴ is lower alkyl or carboxy(lower)alkyl;
R⁵ is amino;
R⁶ and R⁷ are independently guanidino or amino; and
R⁸ and R⁹ are independently amidino, amino, imino(lower)alkylamino or guanidino, or
R⁸ and R⁹, together with the adjacent alkylene(s) and the nitrogen atom, form a group represented by 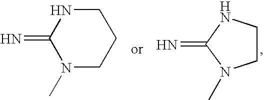
or a pharmaceutically acceptable salt thereof.

5. A process for preparing a compound of the formula [I]: 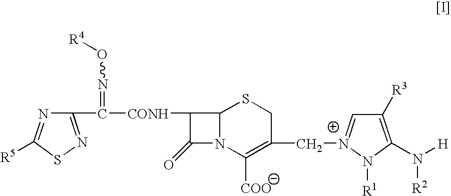
wherein
R¹ is lower alkyl or hydroxy(lower)alkyl, and
R² is hydrogen or amino protecting group, or
R¹ and R² are bonded together to form lower alkylene;
R³ is a group represented by 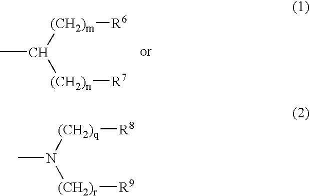
wherein
R⁶ and R⁷ are independently optionally protected amino or optionally protected guanidino, provided that R⁶ and R⁷ are not simultaneously amino groups,
m and n are independently an integer of 0 to 6,
R⁸ and R⁹ are independently optionally protected amino, optionally protected imino(lower)alkylamino, optionally protected guanidino or optionally protected amidino, and
q and r are independently an integer of 0 to 6, or
R⁸ and R⁹, together with the adjacent alkylene(s) and the nitrogen atom, form saturated nitrogen-containing heterocycle optionally having substituent(s),
R⁴ is lower alkyl optionally substituted with carboxy or protected carboxy; and
R⁵ is amino or protected amino,
or a pharmaceutically acceptable salt thereof, which comprises
(1) reacting a compound of the formula [II]: 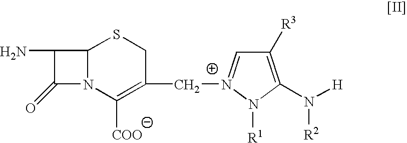
wherein R¹, R² and R³ are each as defined above, or its reactive derivative at the amino group, or a salt thereof with a compound of the formula [III]: 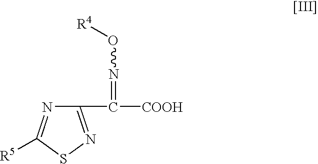
wherein R⁴ and R⁵ are each as defined above, or its reactive derivative at the carboxy group, or a salt thereof to give a compound of the formula [I]: 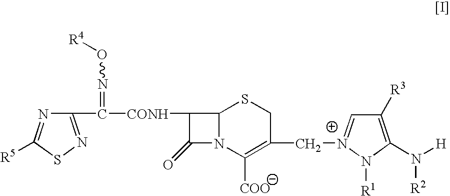
wherein R¹, R², R³, R⁴ and R⁵ are each as defined above, or a salt thereof, or
(2) subjecting a compound of the formula [Ia]: 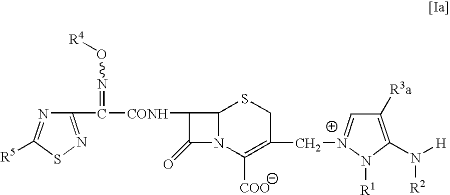
wherein R¹, R², R⁴ and R⁵ are each as defined above, and R³a is a group represented by 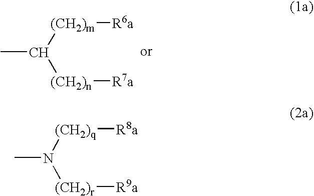
wherein m, n, q and r, are each as defined above,
R⁶a and R⁷a are independently protected amino or protected guanidino, and
R⁸a and R⁹a are independently protected amino, protected imino(lower)alkylamino, protected guanidino or protected amidino, or a salt thereof, to elimination reaction of the amino protecting group to give a compound of the formula [Ib]: 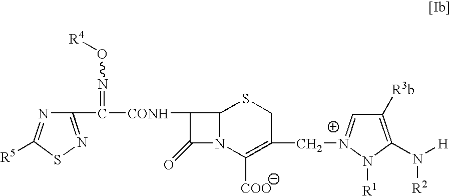
wherein R¹, R², R⁴ and R⁵ are each as defined above, and R³b is a group represented by 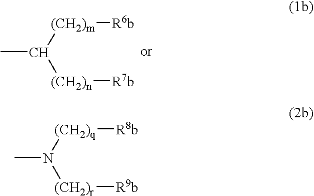
wherein m, n, q and r, are each as defined above,
R⁶b and R⁷b are independently amino or guanidino, and
R⁸b and R⁹b are independently amino, imino(lower)alkylamino, guanidino or amidino, or a salt thereof, or
(3) reacting a compound of the formula [VI]: 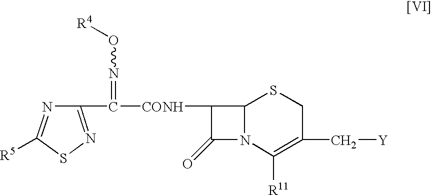
wherein R⁴ and R⁵ are each as defined above, R¹¹ is protected carboxy, and Y is a leaving group, or a salt thereof with a compound of the formula [VII]: 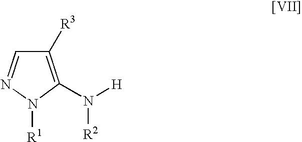
wherein R¹, R² and R³ are each as defined above, or a salt thereof to give a compound of the formula [VIII]: 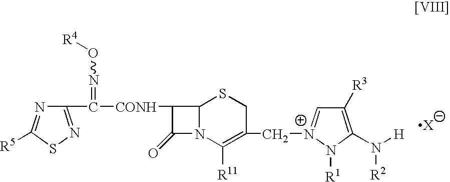
wherein R¹, R², R³, R⁴, R⁵ and R¹¹ are each as defined above, and
X^{\hat{-}} is an anion, or a salt thereof, and
subjecting the compound of the formula [VIII] or a salt thereof to elimination reaction of the carboxy protecting group, to give a compound of the formula [I]: 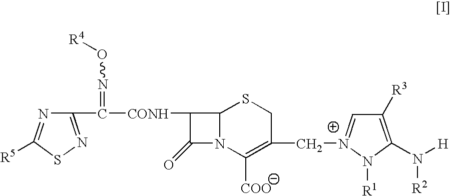
wherein R¹, R², R³, R⁴ and R⁵ are each as defined above, or a salt thereof.

6. A compound of claim 1 or a pharmaceutically acceptable salt thereof for use as a medicament.

7. A compound of claim 1 or a pharmaceutically acceptable salt thereof for use as an antimicrobial agent.

8. Use of a compound of claim 1 or a pharmaceutically acceptable salt thereof for manufacture of a medicament for treating infectious diseases.

9. A pharmaceutical composition comprising a compound of claim 1 or a pharmaceutically acceptable salt thereof in admixture with a pharmaceutically acceptable carrier.

10. A method for the treatment of infectious diseases which comprises administering a compound of claim 1 or a pharmaceutically acceptable salt thereof to a mammal.
